(19) United States
(12) Patent Application Publication
(10) Pub. No.: US 2025/0223558 A1
(43) Pub. Date: Jul. 10, 2025

(54) LARGE SCALE MANUFACTURING OF IPSC DERIVED HSC AND PROGENY

(71) Applicant: Celyntra Therapeutics SA, Mont-Saint-Guibert (BE)

(72) Inventors: Elena Matsa, Charleroi (BE); Benjamin Demoulin, Charleroi (BE); Virginie Claudine Séverine Stygelbout, Charleroi (BE); Stefan Robbert Braam, Charleroi (BE); Eva D'Amico, Charleroi (BE)

(73) Assignee: Celyntra Therapeutics SA, Mont-Saint-Guibert (BE)

(21) Appl. No.: 18/849,557

(22) PCT Filed: Apr. 5, 2023
(86) PCT No.: PCT/EP2023/059036

§ 371 Date: Sep. 23, 2024

(30) Foreign Application Priority Data
Apr. 5, 2022 (EP) 22166735.5
Apr. 5, 2022 (EP) 22166737.1

(51) Int. Cl. C12N 5/0789

(52) U.S. Cl. CPC C12N 5/0647; C12N 2501/115; C12N 2501/125; C12N 2501/145; C12N 2501/155; C12N 2501/165; C12N 2501/2303; C12N 2501/2306; C12N 2501/26; C12N 2506/45

(57) ABSTRACT

This invention describes a method for in vitro production of a population of human hemogenic endothelial cells (HECs), preferably further differentiated to hematopoietic stem cells (HSC). Particularly the method relates to a method of differentiating pluripotent stem cell aggregates to hemogenic endothelial cell (HEC) aggregates, and further differentiating the hemogenic endothelial cell aggregates to hematopoietic stem cell (HSC) aggregates, wherein the step of differentiating PSCs to HECs comprises a step of culturing the cells with BMP4 and a step of culturing the cells without BMP4. The invention also relates to populations of human HSCs or cryopreserved HSCs, as well as a culture medium or a bioreactor with culture medium comprising the cells or population of cells.

### FIELD OF THE INVENTION

This invention pertains in general to method for in vitro production of a population of hemogenic endothelial cell (HEC) and/or human hemopoietic stem cells (HSC). Particularly the method relates to differentiation of pluripotent stem cell aggregates to hemogenic endothelial cell (HEC) aggregates, and further differentiating the hemogenic endothelial cell aggregates to hematopoietic stem cell (HSC) aggregates. The invention further relates to cells comprising a first population of cell aggregates comprising HEC cells and a second population in the form of a suspension of single cells comprising HSC cells. The invention also relates to populations of human HSCs or cryopreserved HSCs, as well as a culture medium or a bioreactor with culture medium comprising the cells or population of cells.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

Cell therapy with immune cells expressing chimeric antigen receptors (CARs) have revolutionized the field of cancer. The most efficacious CAR cell therapy to date is the treatment of patients with highly relapsed/refractory CD19-positive hematological malignancies using CD19-CAR T cells derived from autologous T cells.

Despite the healthcare benefits, several limitations of CAR cell therapies have also been identified, including the challenges associated with leukapheresis, manufacturing, and efficacy, as well as the high cost and slow turn-around time of adoptive cellular therapy (i.e., primary immune cell isolation, with subsequent ex vivo manipulation and delivery into patients as a therapeutic).

To overcome these obstacles, allogeneic CAR strategies are increasingly being implemented and hold the promise of offering quicker, more efficacious, and more affordable cell therapies. A major obstacle for allogeneic therapies is histocompatibility between donor and recipient, which can be mitigated through genome editing approaches to generate “universal” cell donors and circumvent graft vs. host disease (GVHD) where grafted cells recognize host cells as non-self.

The combination of genome editing with hiPSC technologies promises to provide unlimited numbers of “off-the-shelf” allogeneic cells for cell therapy, which can be readily available to patients at reduced cost and without the need for long lead times. To meet the clinical demand for allogeneic cell therapies, manufacturing solutions must evolve to incorporate improvements in use of closed systems (closed culture systems), scalability, GMP compatibility of starting materials and manufacturing processes, efficacy and reproducibility of differentiation of hiPSCs to specialized lineages and quality of the cells obtained.

Tursky et al. (Stem Cell Reports, Vol. 15, 735-748, Sep. 8, 2020) describe a direct comparison of different differentiation methods from human induced pluripotent stem cells in a 2D or 3D culture system. Although direct hematopoietic induction of 2D attached PSCs on specific matrix can be successfully achieved, it would require an extremely large surface area to achieve large scale, commercially valuable production. Since in said direct comparison the 2D culture system provided the best results it is evident that there is an unmet need for alternative, efficient suspension cultures, particularly those that enable upscaling to increased yield (e.g. to industrial levels in bioreactors), and method for providing such.

WO 2020/086889 A1 discloses a 3D culture method of differentiating pluripotent stem cell aggregates to hemogenic endothelial cell (HEC) aggregates, and further differentiating the hemogenic endothelial cell aggregates to hematopoietic stem cell (HSC) aggregates, starting from a plurality of first spheres comprising pluripotent stem cells (PSCs) wherein the step of differentiating PSCs to HECs is performed, among others, in the presence of BMP4 for the entire duration of the step.

WO2022019768 relates to infrastructure and associated manufacturing procedures for large scale manufacturing of PSC derived cells, particularly in a closed systems.

A drawback of methods described in the art is that the methods are generally not very efficient, meaning that both the percentage and absolute number of cells that are differentiated to hematopoietic stem cells is relatively low. Another relevant aspect is that there is need for improved culture methods that allow to further expand the hematopoietic stem cells, either directly after having obtained the cells or after a step of cryopreservation of the obtained cells followed by thawing and culturing, such that the cells maintain their characteristics and that these cells can be further differentiated to specialized lineages such as T cells and/or NK cells.

In light of this, methods, products and compositions with increased efficiency and yield would be highly desirable, but are not yet readily available. In particular, there is a clear need in the art for reliable, efficient and reproducible methods, products and compositions that allow the differentiation of hematopoietic stem cells to be scaled up for therapeutic purposes. Accordingly, the technical problem underlying the present invention can been seen in the provision of such methods, products and compositions for complying with any of the aforementioned needs. The technical problem is solved by the embodiments characterized in the claims and herein below.

### SUMMARY OF THE INVENTION

As embodied and broadly described herein, the present invention is directed to the surprising finding that when differentiating pluripotent stem cells (PSCs) to HECs, the number of HECs can be increased dramatically when starting with aggregates comprising pluripotent stem cells having an average size of about 20-55 micrometers in diameter, preferably less than about 49 micrometers, when differentiating pluripotent stem cells (PSCs) to HECs. It is a further finding that when the differentiation step starting from the aggregated PSCs to HECs is performed in a two-step process, where in the first step BMP4 is added to the medium and in the second step BMP4 is present in a reduced concentration compared to the concentration of BMP4 in the first step, preferably essentially absent in the medium, the formation of definitive hemogenic hematopoiesis is promoted (illustrated by the reduced CD253a expression on the HECs obtained in the methods of the invention) providing for HECs and/or HSCs with high potential for differentiation into both lymphoid and myeloid cell types. Therefore in a first aspect, the invention relates to a method for in vitro production of a population of hemogenic endothelial cells (HECs), comprising:
(a) culturing a suspension of pluripotent stem cells (PSCs), thereby providing a plurality of first cell aggregates comprising said PSCs;
(b) culturing the plurality of first cell aggregates in culture medium to induce differentiation of the PSCs comprised in the plurality of first cell aggregates to generate a plurality of second cell aggregates comprising hemogenic endothelial cells (HECs),
wherein the plurality of first cell aggregates have an average size of about 49 or less micrometers in diameter, and/or
wherein step (b) comprises a step (b1) of culturing wherein the culture medium does comprise a SMAD pathway agonist, preferably BMP4 and a step (b2) of culturing wherein the culture medium is at least partly replaced by fresh medium which does not comprise a SMAD pathway agonist and wherein step (b1) is before step (b2). Preferably the method is performed in a closed culture system, for example in a bioreactor.

In another embodiment, the method comprises step (c) wherein the cells of the second cell aggregates are further differentiated into HSCs using HSC differentiation media described herein.

In a second aspect the invention relates to a composition comprising a first population and a second population of cells, wherein the first population is in the form of cell aggregates comprising HEC cells and wherein the second population is in the form of a suspension of single cells comprising HSC cells, preferably wherein the second population comprises at least 50% HSC cells, or at least 75% HSC cells, or at least 80% or at least 90% HSC, wherein the HSC cells express the marker(s) CD34, CD43 and/or CD45, or wherein the composition comprises of the second population of cells.

In a third aspect the invention relates to a population of human HSCs or cryopreserved HSCs as defined herein.

In a fourth aspect the invention relates to a culture medium or a bioreactor with culture medium comprising the cells or population of cells as defined herein, or comprising cell or aggregates as defined herein.

### BRIEF DESCRIPTION OF THE DRAWINGS

Embodiments of the invention are further described hereinafter with reference to the accompanying drawings, in which:

FIG. 1: Schematic representation of process, biomarkers, and cell culture media with corresponding growth factors and small molecules.

FIG. 2: Optimizing HEC

FIG. 2A: shows examples of iPSC aggregates generated using 2 different iPSC aggregating methodologies. AggreWell plates are a standard academic aggregation technique that works by adding a single cell suspension to the cell culture plates containing microwells, and then centrifuging to distribute the cells evenly in the microwells. Cells form cell-to-cell contacts and aggregate together without adhering to the surface. The bioreactor methodology works by inoculating a single cell suspension in a stirred tank reactor. Small aggregates form spontaneously in the bioreactor, by spontaneous clustering of single cells. The bioreactor methodology is preferred for scalability and does not require manual intervention.

FIG. 2B: applying the iPSC to HEC differentiation process using two different types of aggregate inputs surprisingly leads to ˜5× higher differentiation efficiencies of the CD34+/CD144+/CD73-target cell population with aggregates generated using the bioreactor methodology.

FIG. 2C: applying T-lymphocyte culture conditions on bioreactor derived HEC aggregates plated on vitronectin coated tissue culture plastic in XVIVO15 medium+VEGF 50 ng/ml+huSCF 100 ng/ml+bFGF 10 ng/ml+IL7 20 ng/ml) from day 6 onwards leads to slight upregulation of the lymphoid marker CD1a by day 11.

FIG. 2D: Subsequent culture in the same culture conditions results 3 days later (day 14) in growth of this population to 45%, suggesting effective differentiation to the T-cell lineage ( ).

FIG. 2E: By day 32 more mature T-cell markers CD4 and CD3 are robustly expressed in the released single cells.

FIG. 2F: applying monocyte-cell culture conditions on bioreactor derived HEC aggregates (using either medium 1 XVIVO15+MCSF 100 ng/ml+IL3 25 ng/ml, or medium 2 XVIVO15+TPO 50 ng/ml+Flt3L 10 ng/ml+huSCF 50 ng/ml+MCSF 80 ng/ml+GMCSF 10 ng/ml) from day 6 onwards leads to pure populations of CD11b, CD14, CD45 in the released single cells, example data from day 28 is shown.

FIG. 3: Optimization of Aggregate density at the start of differentiation to improve process robustness

FIG. 3A, B: To further optimize the process, a scaled-down plate based shaker deck system in a standard cell culture incubator was used to test how aggregate density affected the differentiation from iPSC to HEC. iPSC aggregates were formed using bioreactor technology and re-seeded in plates at the start of differentiation. 3 densities where tested, 667 aggregates per ml, 6,667 aggregates per ml and 33,333 aggregates per ml. The lower density of 667 aggregates per ml clearly allows for the most efficient differentiation, evidenced by the highest CD34+/CD144+/CD73-target cell population.

FIG. 4: Optimization of HEC differentiation parameters to enhance HEC to HSC conversion

Hematopoiesis occurs in two waves. The primitive myeloid restricted hematopoiesis and the definitive lymphomyeloid hematopoiesis. CD235a is an important marker that exclusively marks primitive hematopoiesis.

FIG. 4A, B: Further refinement of differentiation conditions using the optimized aggregate methods led to the surprising finding that modulation of the BMP signaling in the period between iPSC and HEC (day 2-6) could significantly reduce CD235a and simultaneously reduce the endothelial marker CD73 without significant effect on the target population CD34+/CD144+/CD73−.

FIG. 4C: Further differentiation of HEC generated by using the two methodologies (with and without BMP4 between days 2-6) resulted in highly pure single cell HSC when exposed for 4-8 days to HSC conditions (IMDM+BSA, ITS-X, β-me, Ascorbic acid-2P, Glutamax, TPO, hSCF, FIT3-L, IL-6 and IL-3). Surprisingly the cells coming from HEC which lacked BMP4 stimulation day 2-6, consistently maintain the CD34+/CD43+/CD45+target cell population around ˜80% whereas the cells coming from the d0-6 continuously exposed BMP4 condition rapidly loose target cell fate and upregulate the myeloid marker CD14.

FIG. 4D: Taking additional markers CD44 and CD14 in consideration, shows that the improved manufacturing method generates HSC with the expected marker profile

FIG. 4E: repetition of the experiments in the bioreactor and the scaled down shaker plate incubator system showed similar results with slightly higher cell yields in the bioreactor

FIG. 4F: single cell HSC differentiated from HEC generated using the process lacking BMP between days 2-6, where collected, resuspended in CryoStor CS10 and successfully cryopreserved in LN2. Cells were successfully thawed with >90% viability and subsequently exposed to proliferation conditions (IMDM+BSA, ITS-X, β-me, Ascorbic acid-2P, Glutamax, TPO, hSCF, FIT3-L, IL-6, IL-3 and UM729). Further culture in these conditions lead to further expansion >10×.

FIG. 4G: applying NK cell culture conditions on bioreactor derived HSC) from day 12 onwards leads to activation of the NK marker CD56, by day 28 of differentiation.

FIG. 5A: Aggregate formation of PSCs (culturing for 3 days (D−3-D0))

FIG. 5B: Aggregate formation of PSCs (culturing for 2 days (D−2-D0))

FIG. 5C: Measurements of CD144/CD73/CD34 markers in HEC differentiated from PSCs following 2 days of first aggregate formation compared to HECs differentiated from PSCs following 3 days of first aggregate formation.

FIG. 6: Marker expression of primitive markers CD235a & KDR (CD309) (in %-ages) per tested condition (2-6, see Table 1). Condition 6 (BMP4 25 ng/ml between D0-D2 and 0 ng/ml between D2-D6) shows reduced CD235 expression in comparison to other tested conditions.

FIG. 7: Cell fold increase per tested condition 2-6 over D0-D6 (see Table 1). High BMP4 concentration (100 ng/mL) results in a poor yield at Day 6, whereas condition 6 shows a good yield at day 6.

FIG. 8: Average cell density of floating cells per mL for each tested condition 2-6 (see Table 1) over D3-D14.

FIG. 9: Marker expression (in %-ages) per tested condition 2-6 (see Table 1) on days 10, 12 and 14. In conditions showing a higher CD235 expression at day 6 (primitive marker) (see FIG. 6), e.g., when BMP4 was added from Day 2, even at low concentration (10 ng/ml), there is expression of myeloid marker (CD14) in HSC at Day 14. Also, high BMP4 concentration (100 ng/ml) results in lower yield of HSC at Day 14.

FIG. 10A: HSPC purity (in CD45/CD43/CD34 expression %-ages), yield (Cell conc. vc/ml) and conversion factor (1 hiPSC to #CD45+/CD43+/CD34+). 4 different media have been tested (see Table 2).

FIG. 10B: Expression %-ages of specific lineage markers CD7 (Lymphoid), CD11b (myeloid), CD41/CD49f.

FIG. 11A: Total cell quantity HSPC on Day 14 relative to condition 11. Conditions 1, 5, 10 and 11 (see Table 4).

FIG. 11B: Cell identity at Day 14 (CD34+ or CD34+/CD7+) for Conditions 1, 5, 10 and 11 (see Table 4).

FIG. 11C: CD11b myeloid marker expression for Conditions 1, 5, 10 and 11 (see Table 4) on Day 14.

FIG. 12A: Total cell quantity CD34+/CD7+ cells on Day 14 relative to condition 11. Conditions 1, 5, 10 and 11 (see Table 4).

FIG. 12B: Floating cell generation (vc/mL). Conditions 1, 5, 10 and 11 (see Table 4).

FIG. 13: Floating cell generation (vc/mL) for conditions 2, 3, 10, 12, 13 (see Table 5).

FIG. 14A: Cell concentration (vc/mL) CD45+/CD43+/CD34+yield for conditions 2, 3, 10, 12, 13 (see Table 5) on days 10, 12, 14.

FIG. 14B: Cell concentration (vc/mL) CD7+yield for conditions 2, 3, 10, 12, 13 (see Table 5) on days 10, 12, 14.

FIG. 14C: Cell concentration (vc/mL) CD11b+yield for conditions 2, 3, 10, 12, 13 (see Table 5) on days 10, 12, 14.

FIG. 15: HSC expansion fold (cumulative) under 6 different post-thawing conditions over time.

FIG. 16: CD45+/CD43+/CD34+expression (in %) of cells exposed to 6 different post-thawing conditions (see Table 6) after 3 and 7 days.

FIG. 17: CD7+lymphoid marker expression (in %) of cells exposed to 6 different post-thawing conditions (see Table 6) after 3 and 7 days.

FIG. 18: CD11b+myeloid marker expression (in %) of cells exposed to 6 different post-thawing conditions (see Table 6) after 3 and 7 days.

FIG. 19: Amount (cell concentration in vc/ml) of CD45+/CD43+/CD34+ cells after 3 and 7 days exposed to different post-thawing conditions (see Table 6 for the details on conditions 2, 5, 6,7 8 and 9).

### DESCRIPTION

### Definitions

A portion of this disclosure contains material that is subject to copyright protection (such as, but not limited to, diagrams, device photographs, or any other aspects of this submission for which copyright protection is or may be available in any jurisdiction.). The copyright owner has no objection to the facsimile reproduction by anyone of the patent document or patent disclosure, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

For purposes of the present invention, the following terms are defined below.

As used herein, the singular form terms “a,” “an,” and “the” include plural referents unless the content clearly dictates otherwise. Thus, for example, reference to “a cell” includes a combination of two or more cells, and the like.

As used herein, “about” and “approximately”, when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed invention. Unless otherwise clear from context, all numerical values provided herein include numerical values modified by the term “about.”

As used herein, “and/or” refers to a situation wherein one or more of the stated cases may occur, alone or in combination with at least one of the stated cases, up to with all of the stated cases.

As used herein, “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc. As used herein, the term “at most” a particular value means that particular value or less. For example, “at most 5” is understood to be the same as “5 or less” e.g. 5, 4, 3, 2, 1, or 0.

As used herein, “comprising” or “to comprise” is construed as being inclusive and open ended, and not exclusive. Specifically, the term and variations thereof mean the specified features, steps or components are included. These terms are not to be interpreted to exclude the presence of other features, steps or components. It also encompasses the more limiting “to consist of”.

As used herein, “conventional techniques” or “methods known to the skilled person” refer to a situation wherein the methods of carrying out the conventional techniques used in methods of the invention will be evident to the skilled worker. The practice of conventional techniques in molecular biology, biochemistry, cell culture, genomics, sequencing, medical treatment, pharmacology, immunology and related fields are well-known to those of skill in the art and are discussed, in various handbooks and literature references.

As used herein, “exemplary” or “for example” means “serving as an example, instance, or illustration,” and should not be construed as excluding other configurations, including those disclosed herein. As used herein, “such as” refers to (a) particular example(s) of the forgoing.

Throughout this disclosure, various aspects of the invention can be presented in a range format. It should be understood that the description in range format is merely for convenience and should not be construed as a limitation on the scope of the invention. The description of a range should be considered to have specifically disclosed all the possible subranges as well as individual numerical values within that range including both integers and non-integers. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 etc. This applies regardless of the breadth of the range.

As used herein, “aggregate”, “aggregation” and “aggregated” in connection to cells refer to one of several main types of cell organization, namely the joining or clustering of a cell with another cell, or cells. Moreover, it does not comprise the joining of a cell with a substrate, commonly referred to as “adherence”. Aggregation of cells is based on cell-cell interactions. Such interactions can be formed between cells through cell surface proteins and are normally present in many biological systems such as tissues, organs and the like. Cell aggregation, when compared to a single cell, enhances survival and functioning of the cell. Aggregation of cells can be induced or maintained in vitro by stirring a culture medium comprising (pluripotent stem) cells. When stirring the aqueous suspension of dispersed cells is discontinued cell aggregates are more likely than single cells to rapidly sink to the bottom (settle) of a culture vessel.

As used herein, “closed culture system” refers to a culturing system comprising a culture vessel and added components that is closed/sealed. Said closed- and/or sealed system typically undergoes sterilization prior to use and after being sealed, thus retaining its sterility. During the use of the culture vessel the integrity of the system is not breached, thus maintaining the sterility of the system. Sterile connections can be made for example using single use sterile connectors or using welding and sealing. The integrity of the system can for example be breached by lifting a cap or a lid, opening a valve or a tube, and the like. As used herein the term “closed system” preferably refers to a closed culturing system comprising a culture bioreactor or culture vessel and its components, including means for mixing culture medium comprised in the culture vessel and means for collecting and replacing medium without breaching sterility. Said bioreactor is used to maintain, culture, grow, differentiate, manipulate a cell culture without a breach of the integrity of the sterility of the closed system. Examples of closed culture systems suitable for the methods provided herein are described in WO2022019768.

As used herein “cryopreservation” refers to a process where organelles, cells, tissues, extracellular matrix, organs, or any other biological constructs susceptible to damage caused by unregulated chemical kinetics are preserved by cooling to very low temperatures. Typically, the biological materials are cooled to −80 degrees Celsius or −196 degrees Celsius, thereby stopping any enzymatic or chemical activity which might cause damage to the biological material. Cryopreservation methods seek to reach low temperatures without causing additional damage caused by the formation of ice crystals during freezing. Cryopreservation allows continued proliferation and/or differentiation of cells after the cryopreserved cells are thawed and provided with a suitable culture medium. A cryopreserved cell is characterized by having a temperature below 0 degrees Celsius, typically below −50, −80, −100, −150 or −180 degrees Celsius.

As used herein, “culturing”, “cultivating”, “growing” or variations thereof refer, when directed to a cell or cells, to a method step to propagate, expand or maintain a population of cells in culture media of various kind. Conventional methods and techniques are well-known to the skilled person in the field of molecular biology, biology, biochemistry, genomics, cell culturing and the like. Although the term “culturing” is generally understood to include the proliferation or division of cells, it also includes methods of differentiating cells in culture medium. Proliferation and differentiation are not mutually exclusive and may happen at the same time in the same culture medium.

The term “culture media” also, and preferably, includes media that are suitable for the in vitro cell culture of human or animal cells for a prolonged period of time. Such culture media comprises sufficient components to allow the cells to grow, proliferate and/or differentiate over longer period of, for example, for example, at least a day. A “defined culture medium” refers to a (growth) medium suitable for the in vitro cell culture of human or animal cells and in which all of the chemical components are known. Such defined media does not or essentially not comprise any ill-defined source of nutrients and/or other ill-defined factors. A defined culture medium may be serum-free.

As used herein, “differentiating” and “differentiation” relate to the progression of a cell further down a developmental pathway within a lineage. Differentiation of cells, including pluripotent stem cells can be induced by means of compounds that direct differentiation of such stem cells within a lineage. Differentiation typically is controlled by the interaction of cellular genes and the chemical and physical surroundings of the cell, usually by means of signaling pathways involving proteins embedded in the cell surface. Within the context of the current invention, the formation of hemogenic endothelial cells starting from pluripotent stem cells, the formation of hematopoietic stem cells from hemogenic endothelial cells, as well as the formation of various cells such as erythroid/megakaryocytic progenitor cells, erythrocytes, megakaryocytes, platelets, common lymphoid progenitor cells, lymphoid lineage cells, lymphocytes, natural killer (NK) cells, common myeloid progenitor cells, common granulomonocytic progenitor cells, monocytes, macrophages, dendritic cells and/or T lymphocytes, such as but not limited to regulatory T (Treg) cells (such as resting Treg cells, activated Treg cells or immunosuppressive Treg cells), helper T cells, cytotoxic T cells, memory T cells, natural killer T cells, mucosal associated invariant T cells and gamma-delta T cells from the hematopoietic stem cells are examples of differentiation.

The process of differentiation according to the invention is induced in the cells, preferably of a human origin, by means of exposure to differentiation-inducing culture media compositions and the methods of the invention. Differentiation-inducing culture media compositions typically contain defined growth factors inducing differentiation of the cells thereby directing differentiation towards a desired cell type. Differentiation in general can be detected by the use of specific differentiation markers the absence of presence thereof, alone or in combinations, can define the developmental stage of a cell.

As used herein, “embryonic stem cells”, abbreviated as ‘ES cells’ or ESC (or if of human origin ‘hES cells’ or ‘hESCs’) refers to stem cells that are derived from the inner cell mass of a blastocyst. The skilled person understands how to obtain such embryonic stem cells, for example as described by Chung (Chung et al (2008) Stem Cell Lines, Vol 2 (2): 113-117), which employs a technique that does not cause the destruction of the donor embryo(s). Various ESC lines are listed in the NIH Human Embryonic Stem Cell Registry.

As used herein, “hematopoietic stem cells” (HSCs) refers to multipotent cells that can develop into all types of blood cells, including myeloid-lineage and lymphoid-lineage cells. HSCs can be found in several organs, such as peripheral blood (PB), bone marrow (BM), and umbilical cord blood (UCB), or may be obtained in vitro from pluripotent stem cells. The immediate progeny of hematopoietic stem cells is believed to be “progenitor” cells, which are capable of giving rise to various cell types within one or more lineages, i.e., the erythroid, myeloid and lymphoid lineages. Hematopoietic stem cells are characterized by the expression of hematopoietic cell surface antigens like CD34, CD43, CD45, CD133 or CD117.

As used herein, “hemogenic endothelial cells” (HECs) refers to specialized vascular endothelial cells with the potential to give rise to hematopoietic stem cells (HSCs) and/or progenitor cells (HPCs) during vertebrate embryogenesis and/or in vitro by differentiation. During this differentiation process, sometimes referred to as endothelial-to-hematopoietic transition (EHT), HECs gradually round up, separate from their neighboring cells and bud off, releasing HSC as single cells in the surrounding. Human markers for HECs include CD31, CD144, CD34, and CD184.

As used herein, “in vivo” refers to an event that takes place in a subject's body; “in vitro” refers to an event that takes places outside of a subject's body. For example, an in vitro assay or method encompasses any assay or method conducted outside of a subject. In vitro assays or methods encompass cell-based assays in which cells, alive or dead, are employed. In vitro assays also encompass a cell-free assay in which no intact cells are employed.

As used herein, “induced pluripotent stem cell” or “iPSC” refers to pluripotent stem cells that are derived from a cell that is not a pluripotent stem cell (i.e., from a cell which is differentiated relative to a pluripotent stem cell). Induced pluripotent stem cell can be derived from multiple different cell types, including terminally differentiated cells. Induced pluripotent stem cells generally have an embryonic stem cell-like morphology, growing as flat colonies with large nucleo-cytoplasmic ratios, defined borders and prominent nuclei. In addition, induced pluripotent stem cell may express one or more key pluripotency markers known by one of ordinary skill in the art. To generate induced pluripotent stem cells, somatic cells may, for example, be provided with reprogramming factors (e.g., OCT3/4, SOX2. KLF4, MYC, NANOG, LIN28, etc.) known in the art to reprogram the somatic cells to become pluripotent stem cells.

As used herein, the term “marker” is used to describe the characteristics and/or phenotype of a cell. Markers can be used for selection of cells comprising characteristics of interests. Markers will vary with specific cells. Markers are characteristics, whether morphological, functional or biochemical characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological markers include shape, size, and nuclear to cytoplasmic ratio. Examples of functional markers include the ability to migrate under particular conditions and the ability to differentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

As used herein, “pluripotent stem cell” or “PSC” refers to a stem cell capable of producing a broad range or large variety of cell types of the organism and can produce cells of the germ layers, e.g., endoderm, mesoderm, and ectoderm, of a mammal and encompasses at least pluripotent embryonic stem cells and induced pluripotent stem cells. Pluripotent stem cells can be obtained in different ways. Induced pluripotent stem cells (iPSCs) may, for example, be derived from somatic cells. Pluripotent stem cells may also be in the form of an established cell line. According to an embodiment, PSCs for use in a method according to the invention are derived from human cells or tissue, e.g. iPSCs for use in the methods described herein may be derived from human cells, such as human somatic cells. Such human PSCs or human iPSCs may also be referred to as hPSCs or hiPSCs respectively. Progeny of PSCs or iPSCs (e.g. HECs or HSCs) may equally be attributed the prefix “human” or “h” when derived from cells of human origin and are equally considered for the methods described herein.

PSCs, such as iPSCs, for use according to the invention may be further modified e.g. by transfection with an expression vector encoding a functional protein, such as but not limited to an expression vector encoding a chimeric antigen receptor, a signaling peptide, a peptide required by a subject in need of protein substitution therapy to replace (genetic) defective, aberrant or absent functional protein in said subject.

As used herein, “proliferating” and “proliferation” relate to an increase (growth) in the number of cells in a population by cell division, i.e., cells undergoing mitosis. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

As used herein, “stem cells” refer to a population of undifferentiated cells defined by their ability at the single cell level to both self-renew and differentiate to produce progeny cells, including self-renewing progenitors, non-renewing progenitors, and terminally differentiated cells (Morrison et al. (1997) Cell 88:287-298).

### DETAILED DESCRIPTION

The embodiments of the invention are defined herein. It is contemplated that any method, use or composition described herein can be implemented with respect to any other method, use or composition described herein. Embodiments discussed in the context of methods, use and/or compositions of the invention may be employed with respect to any other method, use or composition described herein. Thus, an embodiment pertaining to one method, use or composition may be applied to other methods, uses and compositions of the invention as well.

Any references in the description to methods of treatment refer to the compounds, pharmaceutical compositions and medicaments of the present invention for use in a method for treatment of the human (or animal) body by therapy.

As embodied and broadly described herein, the present invention is directed to the improved methods of differentiating pluripotent stem cells to hemogenic endothelial cells (HECs) (step (a) and step (b)), and/or HECs (further) differentiated to hemopoietic stem cells (step (c)). The method is for inducing differentiation of pluripotent stem cells towards HECs, which according to a further embodiment are cultured to hematopoietic stem cells (HSCs) and/or for manufacturing of such differentiated pluripotent stem cell derived HECs, and/or such differentiated pluripotent stem cell derived HSCs. The method may for example be performed in a closed culture system, in particular without intermediate cell selection step. The method allows vast amounts of such differentiated cells (i.e. HECs and/or HSCs) to be manufactured. The method allows for high output of cells over input of cells ratio's (e.g., expressed by number of cells). The method also allows for the production of HSCs that can be further expanded several-fold (e.g. 2, 3, 5, 10, 20-fold or more) directly or after cryopreservation and thawing, and subsequently be differentiated towards specialized lineages as described herein (e.g. using differentiation protocols as described in the art).

The invention takes advantage of existing 3D cell culture protocols for differentiation of pluripotent stem cells. The invention provides an improved method, allowing to increase the efficiency and yield of the method to produce HECs and/or HSCs. The efficiency of differentiation of pluripotent stem cells to HECs, and/or differentiation of HECs to HSCs, relates, for example, to the percentage of cells in the aggregate ultimately becoming an HSC and consequently also the percentage of cells not becoming an HSC, e.g., by differentiating to a different (unwanted or less desirable) cell type. The yield as measured in absolute cell numbers (HECs and/or HSCs) produced by differentiation of the pluripotent stem cell aggregates as defined for the methods herein depends on the efficiency of the differentiation process, and further on the proliferation of the cells during the proliferation protocol. In particular, it was found that the current method for in vitro production of HECs and/or HSCs can suitably be used in high volume closed culture systems, for example using culture vessels such as a bioreactor, a tank, or any other device suitable for the culturing of cells. For example, the volume of the culture vessel can be any volume but is preferably between 2-150 liters, or between 2-100 liters, or between 2-50 liters in volume and/or allows for cultivation in such volumes of culture medium. For example, the method of the current invention allows for cultivation in at least 2, 3, 5, 8, 10, 20, 50 liters of culture medium. In a further embodiment, the efficiency relates to the marker profile of the HECs and/or HSCs resulting from the method. The marker profile of the HECs and/or HSCs will in turn determine the potential of the cells for further differentiation in specified hematopoietic cell types. The differentiation protocol of the aggregated PSCs into HECs as described herein for step (b) promotes definitive lympho-myeloid hematopoiesis. The formation of (high purity) definitive hematopoiesis HECs and/or HSCs as described herein, allows for efficient differentiation of the HECs and/or HSCs into lymphoid or myeloid cell types downstream, thereby reducing the need for intermediate cell-type based isolation and/or selection steps.

Therefore in a first aspect, the invention relates to a method for in vitro production of a population of hemogenic endothelial cells (HECs), comprising:
(a) culturing a suspension of pluripotent stem cells (PSCs) thereby providing a plurality of first cell aggregates comprising said PSCs;
(b) culturing the plurality of first cell aggregates in culture medium to induce differentiation of the PSCs comprised in the plurality of first cell aggregates to generate a plurality of second cell aggregates comprising hemogenic endothelial cells (HECs), wherein
the plurality of first cell aggregates have an average size of about 49 or less micrometers in diameter, and/or
wherein step (b) comprises a step (b1) of culturing wherein the culture medium does comprise a SMAD pathway agonist, preferably BMP4, and, a step (b2) of culturing wherein the culture medium is at least partly replaced by fresh medium which does not comprise a SMAD pathway agonist and wherein step (b1) is before step (b2).

In some embodiments, the fresh medium in step (b2) is not supplemented with a SMAD pathway agonist.

In one embodiment, the invention relates to a method for in vitro production of a population of hemogenic endothelial cells (HECs), comprising:
(a) culturing a suspension of pluripotent stem cells (PSCs) thereby providing a plurality of first cell aggregates comprising said PSCs;
(b) culturing the plurality of first cell aggregates in culture medium to induce differentiation of the PSCs comprised in the plurality of first cell aggregates to generate a plurality of second cell aggregates comprising hemogenic endothelial cells (HECs), wherein
the plurality of first cell aggregates have an average size of about 49 or less micrometers in diameter.

In another embodiment, the invention relates to a method for in vitro production of a population of hemogenic endothelial cells (HECs), comprising:
(a) culturing a suspension of pluripotent stem cells (PSCs) thereby providing a plurality of first cell aggregates comprising said PSCs;
(b) culturing the plurality of first cell aggregates in culture medium to induce differentiation of the PSCs comprised in the plurality of first cell aggregates to generate a plurality of second cell aggregates comprising hemogenic endothelial cells (HECs), wherein step (b) comprises a step (b1) of culturing wherein the culture medium does comprise a SMAD pathway agonist, preferably BMP4, and, a step (b2) of culturing wherein the culture medium is at least partly replaced by fresh medium which does not comprise a SMAD pathway agonist and wherein step (b1) is before step (b2).

In another embodiment, the invention relates to a method for in vitro production of a population of hemogenic endothelial cells (HECs), comprising:
(a) culturing a suspension of pluripotent stem cells (PSCs) thereby providing a plurality of first cell aggregates comprising said PSCs;
(b) culturing the plurality of first cell aggregates in culture medium to induce differentiation of the PSCs comprised in the plurality of first cell aggregates to generate a plurality of second cell aggregates comprising hemogenic endothelial cells (HECs), wherein
the plurality of first cell aggregates have an average size of about 49 or less micrometers in diameter, and
wherein step (b) wherein step (b) comprises a step (b1) of culturing wherein the culture medium does comprise a SMAD pathway agonist, preferably BMP4, and, a step (b2) of culturing wherein the culture medium is at least partly replaced by fresh medium which does not comprise a SMAD pathway agonist and wherein step (b1) is before step (b2).

It is the general understanding in the field of stem cell differentiation, that in order to differentiate pluripotent stem cells to HECs (and optionally further to HSCs) the presence of a SMAD pathway agonist, preferably BMP4, in the culture medium is required in the differentiation step from PSCs to HECs. It was now surprisingly found by the inventors that the yield and efficiency of hematopoietic stem cell production can be increased unexpectedly when only providing a SMAD pathway agonist, preferably BMP4, in the culture medium during part of the differentiation step from PSCs to HECs. In particular it was found that a first step (b1) of culturing wherein a SMAD pathway agonist, preferably BMP4, is added to the culture medium and a subsequent step (b2) of culturing wherein no a SMAD pathway agonist is added to the culture medium differentiation efficiency and yield are increased. For example, step (b1) may be performed for 1, 2, 3, or 4 days, preferably about 2 days, followed by performing step (b2) for 1, 2, 3, 4, 5 or 6 days, preferably about 4 days. In a further embodiment step (b) overall is performed for about 6 days.

Further it was surprisingly found that the yield and efficiency of HEC and/or subsequent HSC production can be increased unexpectedly when carefully controlling first cell aggregate size, in particular starting with aggregates comprising pluripotent stem cells having an average size of about 49 or less micrometers in diameter.

In particular, differentiation towards HECs and/or HSCs consist of a delicate and balanced involvement of different (signaling) pathways that are switched-on or switched-off at different stages of differentiation and, without being bound by theory, the inventors believe that by initiating differentiation of PSCs when forming aggregates having an average size of about 49 or less micrometers in diameter, the cells within the aggregate can more efficiently proliferate and differentiate towards HEC and subsequently towards HSC as defined and described herein, thereby providing for a new and robust method of producing a population of cells comprising HECs and/or HSCs in high amount, absolute and/or relative (purity), which HSCs can efficiently be further proliferated (or expanded) directly or after cryopreservation and thawing. Surprisingly, it was found that the HSCs obtained by the methods described herein can be expanded several fold, as discussed above, while substantially maintaining its HSC phenotype, thus allowing the subsequent differentiation of the HSCs towards more specialized cell lines in high number, high purity and/or with high yield.

In a further embodiment, the invention relates to a method comprising step (a) culturing a suspension of pluripotent stem cells (PSCs) thereby providing a plurality of first cell aggregates comprising said PSCs; step (b) culturing the plurality of first cell aggregates in culture medium to induce differentiation of the PSCs comprised in the plurality of first cell aggregates to generate a plurality of second cell aggregates comprising hemogenic endothelial cells (HECs), wherein step (b) comprises a step (b1) of culturing wherein the culture medium does comprise a SMAD pathway agonist, preferably BMP4, and, a step (b2) of culturing wherein the culture medium is at least partly replaced by fresh medium which does not comprise a SMAD pathway agonist and wherein step (b1) is before step (b2); and step (c) where in step (c1) the plurality of second cell aggregates are cultured in a culture medium to induce differentiation of the HECs comprised in the plurality of second cell aggregates to generate a plurality of third cell aggregates producing hematopoietic stem cells (HSCs) and allowing the HSCs to release (through budding) from the plurality of third cell aggregates in the culture medium to obtain a population of HSCs thereby forming a single cell population of comprising HSCs in suspension in the culture medium.

Further embodiments for executing steps (a), (b) and (c) in accordance with the present invention are provided herein below and may be combined independently to provide for a specific embodiment of the method of the invention.

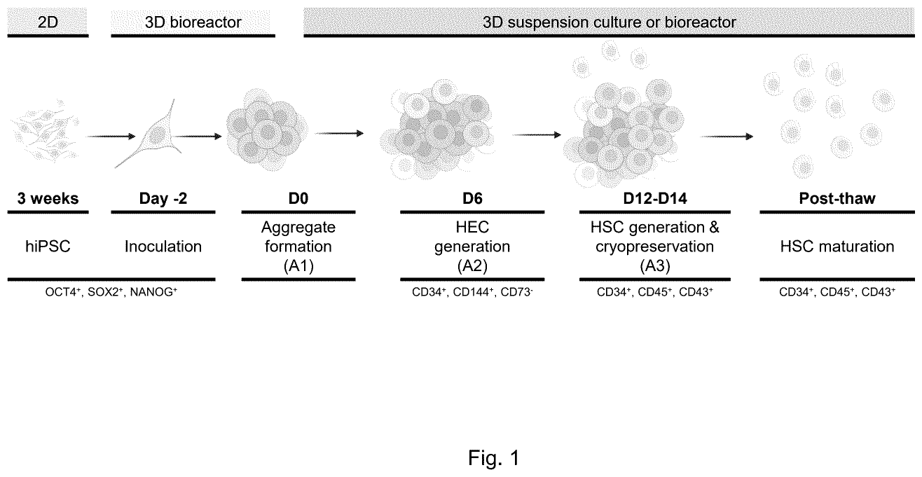

A non-limiting exemplary scheme for differentiation of pluripotent cells to HECs and/or to HSCs can be found in FIG. 1. For example, pluripotent stem cells, such as induced pluripotent stem cells, can be cultured under appropriate conditions. Suitable culture conditions for expanding pluripotent stem cells are known to the skilled person. For example, mTeSR1 medium may be used with Vitronectin culture plate coating and passaging with Accutase, but the skilled person is aware of suitable other media and/or supplements. Cells obtained from the undifferentiated pluripotent stem cell culture can be used to inoculate a 3D culture, for example in a bioreactor. After inoculation, cell aggregates of pluripotent stem cells will start to form. Aggregate formation can be induced by selecting appropriate culture conditions, for example, and in a preferred embodiment, StemMACS™ iPS-Brew XF medium can be used, for example in the presence of, preferably a ROCK inhibitor such as, preferably Y-27632. The skilled person knows and understands that in addition to those described herein other suitable culture media such as mTeSR1, Essential 8, TeSR E8, and or Nutristem media may likewise be used. The skilled person also knows and understands that alternative supplements, for example other ROCK inhibitors, such as blebbistatin, Fasudil, pinacidil, thiazovivin, chroman1 and/or molecules with similar pro-survival potency may be used, for example emricasan, fasudil, thiazovivin, Q-VD-Oph, polyamines and or Trans-ISRIB. After aggregate formation the aggregates comprising pluripotent stem cells are differentiated to HEC comprising aggregates, and in a further embodiment followed by further differentiation towards aggregates producing HSCs. Individual HSCs will be released from the aggregates and can be isolated, for example for cryopreservation and later use, e.g. further expansion and subsequent differentiation.

In step (a), a plurality of first cell aggregates comprising pluripotent stem cells (PSCs) are provided. Embodiments are provided herein further defining the first cell aggregates for differentiation into HEC in accordance with the methods of the present disclosure. In some embodiments the plurality of first cell aggregates have an average size of 49 or less micrometers in diameter.

Depending on the number of pluripotent stem cells introduced, it may be decided to first allow the pluripotent stem cells to proliferate for a certain period of time in order to obtain a desirable number of pluripotent stem cells (aggregates), for example until the majority of the first cell aggregates have the desired average size in diameter. As mentioned above, the culture medium may be any suitable culture medium for proliferation of the pluripotent stem cells, such as commercially available mTeSR1, StemMACS™ iPS-Brew XF, Essential 8, TeSR E8, mTeSR Plus and/or Nutristem media. In (preferred) embodiments wherein the pluripotent stem cells are initially introduced as a single cell suspension, the single cells will form aggregates during cultivation in the culture medium for proliferation of the stem cells (aggregation will typically start within a few hours, for example after 2-3 hours).

In an embodiment the PSCs that are comprised in the plurality of first cell aggregates express the markers OCT3/4, SOX2, and NANOG in at least 80% of cells. In an embodiment the PSCs are induced pluripotent stem cells.

In some embodiments, the plurality of first cell aggregates have an average size about 20-250 micrometers in diameter, or have an average size of about 20-150 micrometers, preferably have an average size of about 20-55 micrometers or have an average size of about 25-50 micrometers or have an average size of about 30-40 micrometers.

In some embodiments, the first cell aggregates have an average size of about 49 or less micrometers in diameter. In one non-limiting example, an average size of about 20-55 micrometers, or an average size of about 49 or less micrometers in diameter can be achieved approximately 2 days after inoculation, for example between 12 and 96 hours after inoculation, preferably between 24 and 72 hours after inoculation, typically resulting in a density of about 110,000 aggregates per ml. The skilled person knows and understands how to determine the diameter of a cell aggregate, for example using methods as described herein.

In addition, in an embodiment the plurality of first cell aggregates have an average size of about 25-50 micrometers or have an average size of about 30-45 micrometers.

It was surprisingly found that in the steps of the method that the yield and efficiency of hematopoietic stem cell production can be increased unexpectedly when starting with aggregates comprising pluripotent stem cells having an average size of about 20-55 micrometers in diameter, preferably having an average size of about 49 micrometers or less, or, about 45 micrometers or less.

In particular differentiation towards HECs and further to HSCs consist of a delicate and balanced involvement of different (signaling) pathways that are switched-on or switched-off at different stages of differentiation and, without being bound by theory, the inventors believe that by selecting aggregates comprising pluripotent stem cells having an average size of about 20-55 micrometers in diameter, -preferably having an average size of about 49 or less micrometers in diameter or, about 45 micrometers or less, the cells within the aggregate can efficiently proliferate and differentiate towards HEC and subsequently towards HSC as defined and described herein, thereby providing for a new and robust method of producing a population of cells comprising HSCs in high amount, both absolute and relative, which HSCs can efficiently be further proliferated (or expanded) directly or after cryopreservation and thawing. Surprisingly, it was found that the HSCs can be expanded several fold, as discussed above, while substantially maintaining a HSC phenotype, thus allowing the subsequent differentiation of the HSCs towards more specialized cell lines in high number and with high yield.

For example, the aggregate size of the plurality of first cell aggregates comprising PSCs may be 20, 21, 22, 23, 23, 24, 25, 27, 28, 29 or 30 micrometers or more, or the aggregate size of the plurality of first cell aggregates comprising PSCs may be 55, 54, 53, 52, 51, 50, 49, 48, 47, 46, or 45 micrometer or less, preferably of about 49, 48, 47, 46, or 45 or less micrometers in diameter. Since the aggregates are substantially spherical, the size when used herein refers to the diameter, wherein the diameter is defined as the longest straight line between two points on the surface of the aggregate. Further, since the methods and compositions refer to plurality of aggregates, it is understood that aggregate size refers to the average size of the aggregates in the culture medium. It is therefore understood that a certain percentage, e.g., 25%, 20%, 15%, 10%, preferably 5%, or less of the aggregates may be either larger or smaller than the indicated size ranges. Therefore, the average aggregate size of the plurality of first cell aggregates comprising PSCs may be between 20 and 55 micrometers, preferably between 22-52, 25-50, 25-49, 28-48, or 30-45 micrometers.

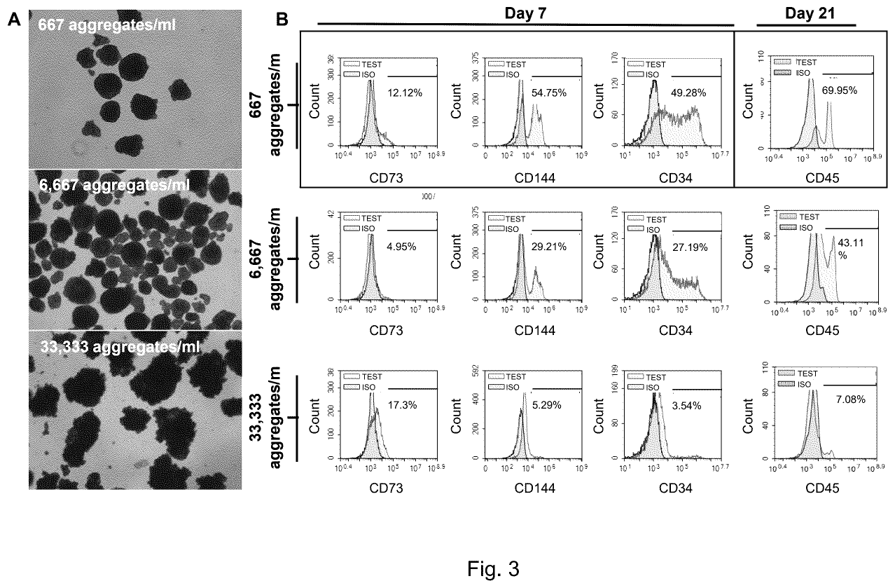

The inventors further surprisingly found that the aggregate density as measured in aggregates per ml may further impact the efficiency of differentiation as described herein. In FIG. 3 representative data is shown, comparing different experiments using three different aggregate densities of 667, 6667 and 33333 aggregates per ml. The aggregates were differentiated to HSCs using otherwise identical protocols, and aggregates were tested for the markers CD73, CD144 and CD34 at day 7, and CD45 at day 21. A substantial increase in differentiation efficiency can be observed when lowering the aggregate concentration from 33333 to 6667 aggregates as can be observed from FIG. 3B, where differentiation under conditions of 6667 or 667 aggregates per ml resulted in less CD73 positive cells and more CD144 and CD34 positive cells at day 7, and more CD45 positive cells at day 21, when compared to differentiation at 33333 aggregates per ml.

Therefore, in an embodiment the plurality of first cell aggregates are provided in the first culture medium at a density of at least 100 aggregates/ml, preferably between 100-100000 aggregates/ml. For example, the plurality of first cell aggregates are provided in the first culture medium at a density of at least 100, 150, 200, 250, 300, 350, 400, 450, or 500 aggregates/ml, and/or 100000, 90000, 80000, 70000, 60000, 50000, 40000, 30000, 25000, 20000, 18000, 16000, 15000, 14000, 13000, 12000, 11000, 10000, 9000, or 8000 aggregates/ml or less. Therefore, the plurality of first cell aggregates are provided in the first culture medium at a density of between 100-100000, 150-60000, 200-20000, 250-10000, 250-8000, 250-6000, 250-4000, or 250-2000 aggregates/ml.

In step (b) the plurality of first cell aggregates are cultured in culture medium to induce differentiation of the PSCs comprised in the plurality of first cell aggregates to generate a plurality of second cell aggregates comprising hemogenic endothelial cells (HECs). In some embodiments, step (b) comprises a step (b2) of culturing wherein the culture medium does not comprise a SMAD pathway agonist such as BMP4, preferably wherein step (b) comprises a step (b1) of culturing wherein the culture medium does comprise a SMAD pathway agonist, preferably BMP4, and, a step (b2) of culturing wherein the culture medium is at least partly replaced by fresh medium which does not comprise a SMAD pathway agonist, preferably BMP4, and wherein step (b1) is before step (b2).

During step (b) the pluripotent stem cells in the first cell aggregates are differentiated to at least in part become HECs. Suitable culture conditions for differentiating pluripotent stem cells to HECs are known to the skilled person. For example, IF9S medium may be used, where IF9S is a IMDM/F 12 based medium plus nine supplements, as described in Uenishi et al., Stem Cell Reports. 2014 Dec. 9; 3 (6): 1073-1084. Generally the culture medium is further supplemented with suitable growth factors such as but not limited to VEGF (e.g. between 5 and 125 ng/mL, preferably about 25 ng/mL), bFGF (e.g. between 5 and 125 ng/ml, preferably about 25 ng/ml), BMP4 (e.g. between 5 and 125 ng/ml, between 5 and 50 ng/ml, between 10 and 40 ng/ml, between 10 and 30 ng/mL, or preferably about 25 ng/ml), CHIR99021 (e.g. between 0.6 and 15 μM, preferably about 3 μM), and SB431542 (e.g. between 0.6 and 15 μM, preferably about 3 μM). Non-limiting examples of other suitable growth media for step (b) are StemPro, Hams-F12, APEL and RPMI. Non limiting examples of suitable supplements are KO-SR, N-2 supplement, B-27 supplement.

In some embodiments, during step (b) the culture medium comprises VEGF and/or bFGF, e.g. throughout step (b) including step (b1) and (b2).

Non-limiting examples of other suitable growth factors for step (b) are Wnt3a, SCF, FIt3-L, TPO.

In an embodiment step (b) is performed for about 6 days, such as for example 3, 4, 5, 6, 7, 8, 9, or 10 days or more. Preferably step (b) is performed for 2-10 days, preferably 3-8 days, or, preferably 5-6 days.

In an embodiment of the method of the invention the plurality of first cell aggregates were obtained by suspension culture in stirred tank bioreactors. In an embodiment the plurality of second cell aggregates are present in the second culture medium at a density of 200,000-800,000 cells/ml. In an embodiment wherein at least 10-50% of the cells in the plurality of second cell aggregates are HECs, preferably at least 20%, 30%, 40%, 50%, 60%, 70%, 80% or more.

In an embodiment the culture medium during step (b) is replaced every day, every other day, every 3rd day, every 4th day, or combinations thereof (e.g., the first replacement is after 1 day and a subsequent replacement is after 2 days). In a particular preferred embodiment, the culture medium during step b) is replaced or refreshed every day.

In some embodiments, during step (b), including step (b1) and (b2) as explained below, at least a part of the medium, preferably all medium, is replaced or refreshed. However, it is noted that it was surprisingly found that it is possible to design robust and reproducible manufacturing processes yielding well-differentiated cells in high quantities in and using a preferably closed culture systems by not collecting all of the culture medium from the culture vessel and replacing it with fresh culture medium, but by collecting only part of the culture medium from the culture vessel before a subsequent, fresh, culture medium (and, where required, growth factors such as those disclosed herein) is added to the remaining culture medium in the culture vessel (and thus wherein part of the previous medium is mixed with the subsequent medium). For example, in one embodiment at most 95 vol. %, 90 vol. %, 85 vol. %, or 80 vol. % of the culture medium in the culture vessel is collected to enable efficient switching of media composition and/or harvesting of (single) cells or material secreted by the cells into the culture medium. For example, in this embodiment, in case the culture vessel comprises 10 liters of culture medium, at most 9500, 9000, 8500 or 8000 milliliter of the culture medium is collected from the culture vessel.

At the same time, it was found that, preferably, at least 30 vol. %, 40 vol. % 50 vol. %, 60 vol. % or 70 vol. % of the culture medium is collected from the culture vessel. Therefore, in some embodiments between, for example between 30 vol. %-95 vol. %, or between 40 vol. % and 95 vol. % or between 50-95 vol. %, or between 60-90 vol. % or between 60-80 vol. %, or between 70-90 vol. % or between 70-80 vol. % of the culture medium is collected from the culture vessel and, preferably, replaced or refreshed with new culture medium. As will be understood by the skilled person, the amount or percentage of medium collected may vary between different moments of collection medium according to the method of the invention. For example, during a first medium collection during step (b) 70 vol % of the culture medium in the culture vessel may be collected and replaced, whereas during a subsequent medium collection during step (b), the same, more or less (e.g., 50 vol. % or 75 vol. %) may be collected and replaced. In some embodiments growth factors are added directly provided to culture medium already present in the culture vessel or closed culture system, without the substantial replacement of old culture medium or the substantial addition of new culture medium (e.g., fresh medium).

In some embodiments between step (b1) and (b2) at least 70 vol. %, 80 vol. %, 90 vol. % or 95 vol. % of the medium is replaced by fresh medium. In some embodiments partly replacing the culture medium comprises replacing at least 30 vol. %, at least 40 vol. %, at least 50 vol. %, at least 60 vol. %, 70 vol. %, 80 vol. %, 90 vol. % or 95 vol. % of the medium by fresh medium.

In an embodiment the duration of step (b2) is more than 50% of step (b) and/or wherein the duration of step (b1) is less than 50% of step (b), preferably wherein step (b2) is for at least two, three or four days and/or wherein step (b1) is for at most one, two, three or four days. For example, step (b1) is 1 day and step (b2) is 2 days, step (b1) is 1 day and step (b2) is 3 days, step (b1) is 1 day and step (b2) is 4 days, step (b1) is 1 day and step (b2) is 5 days, step (b1) is 1 day and step (b2) is 6 days, step (b1) is 2 days and step (b2) is 3 days, step (b1) is 2 days and step (b2) is 4 days, step (b1) is 2 days and step (b2) is 5 days, step (b1) is 2 days and step (b2) is 6 days, step (b1) is 3 days and step (b2) is 4 days, step (b1) is 3 days and step (b2) is 5 days, step (b1) is 3 days and step (b2) is 6 days, step (b1) is 3 days and step (b2) is 7 days, step (b1) is 4 days and step (b2) is 5 days, step (b1) is 4 days and step (b2) is 6 days, or step (b1) is 4 days and step (b2) is 7 days.

In some embodiments the duration of step (b2) is more than 50% of step (b) and/or wherein the duration of step (b1) is less than 50% of step (b), preferably wherein step (b2) is for at least two, three or four days (such as 4 days) and/or wherein step (b1) is for at most three, two or one day (such as 2 days).

It is understood that BMP4 is a TGF-beta family growth factor, therefore it is envisioned that in step (b) the culture medium may be supplemented with an alternative TGF-beta family growth factor, or a SMAD pathway agonist. In such case, in step (b1) preferably the cells are cultured in medium comprising a TGF-beta family growth factor, or a SMAD pathway agonist. Therefore, in an embodiment, culturing the plurality of first cell aggregates in step (b) comprises a step (b2) of culturing wherein no TGF-beta family growth factor, or a SMAD pathway agonist is added to the medium, preferably wherein culturing the plurality of first cell aggregates in step (b) comprises a step (b1) of culturing wherein the culture medium does comprise a TGF-beta family growth factor, or a SMAD pathway agonist, and/or a GSK3 inhibitor and a step (b2) of culturing wherein no TGF-beta family growth factor, or a SMAD pathway agonist, and/or a GSK3 inhibitor is added to the medium, and wherein step (b1) is before step (b2).

The TGF-beta family growth factor may be selected from TGFB1, TGFB2, TGFB3, BMP1, BMP2, BMP3, BMP4, BMP5, BMP6, BMP7, BMP8a, BMP8b, BMP10, BMP11, BMP15, GDF1, GDF 2, GDF 3, GDF 5, GDF 6, GDF8, GDF9, GDF10, 11, GDF15, INHA, INHBA, INHBB, INHBC, INHBE, Activin A, Activin AB, Activin B, LEFTY1, LEFTY2, MSTN, NODAL, NRTN, PSPN, AMH, ARTN, or combinations thereof. Alternatively, the TGF beta pathway may also be activated by small molecule agonists such as but not limited to SRI-011381 hydrochloride. The GSK3 inhibitor may be selected from beryllium cations, copper cations, lithium cations, mercury cations, tungsten cations, 6-BIO, Dibromocantharelline, Hymenialdesine, Indirubin, Meridianin, CHIR99021, CT98014, CT98023, CT99021, TWS119, SB-216763, SB-41528, AR-A014418, AZD-1080, Alsterpaullone, Cazpaullone, Kenpaullone, Manzamine A, Palinurine, Tricantine, TDZD-8, NP00111, NP031115, Tideglusib, HMK-32, L803-mts, L807-mts, COB-187, COB-152, or combinations thereof.

In further embodiments during at least part of step (b2) the cells are cultured in the presence of a TGF-beta pathway inhibitor, such as SB431542 (e.g. between 0.6 and 15 μM, preferably about 3 μM). Alternatives to SB431542 contemplated for use in step (b2) of the methods herein include other Activin/Nodal/TGF-beta pathway inhibitors known to the skilled person such as A 83-01.

In further embodiments also during at least part of step (b2) the cells are also cultured in the presence of a GSK3 inhibitor, preferably a GSK3-beta inhibitor, such as CHIR99021 (e.g. between 0.6 and 15 μM, preferably about 3 M) or combinations thereof. Alternatives to CHIR99021 contemplated for use in step (b2) of the methods herein include other GSK3-beta inhibitors or more in general other compounds known to the skilled person to activate the Wnt pathway such as but not limited to the list of compounds of Table 1 as published by Bonnet C. et al. 2021 (“Wnt signaling activation: targets and therapeutic opportunities for stem cell therapy and regenerative medicine” RSC Chem Biol. 2021 Aug. 5; 2 (4): 1144-1157).

As explained above for step (b) as part of step (b1) and (b2) all medium may be replaced or refreshed. However, it is surprisingly found that it is possible to design robust and reproducible manufacturing processes yielding well-differentiated cells in high quantities in closed culture systems by not collecting all of the culture medium from the culture vessel and replacing it with fresh culture medium, but by collecting only part of the culture medium from the culture vessel before a subsequent, fresh, culture medium (and, where required, growth factors such as those disclosed herein) is added to the remaining culture medium in the culture vessel (and thus wherein part of the previous medium is mixed with the subsequent medium). It is understood that in these embodiment residual medium is still present, for example in some culture conditions up to 20% or more of the medium may be left when adding fresh medium. It is understood that the residual medium (e.g. of step (b1)) may still comprise some BMP4 (or TGF-beta family growth factor, or a SMAD pathway agonist), therefore when referring herein to “cultured in medium comprising no BMP4” or “culturing wherein no BMP4 is added to the culture medium” (or TGF-beta family growth factor, or a SMAD pathway agonist) it is meant that in that step the new medium added does not comprise BMP4 (or TGF-beta family growth factor, or a SMAD pathway agonist). In other words, the step (b2) comprises replacement of all or part of the medium comprised in the culture system with fresh medium that does not comprise BMP4. In case only part of the medium in the culture system is replaced or refreshed, residual old culture medium is still present. Unexpectedly it was found that by including a step (b2) differentiation efficiency and yield are increased. In particular it was found, as shown in the examples, that the HSCs thus obtained, for example after cryopreservation and thawing can efficiently be expanded without substantially losing their phenotypical characteristics (see Examples).

In some embodiments the culture medium used to cultivate according to step (b2) comprise less than (residual) 10 ng/ml, preferably less than 5, 4, 3, 2, 1 ng/ml or 0 ng/ml BMP4.

In some embodiments, the SMAD pathway agonist is BMP4 and the concentration of BMP4 in the culture medium of step (b1) is between 10 and 40 ng/ml, e.g. between 10 and 30 ng/ml or about 25 ng/ml, and/or, the (residual) concentration of BMP4 in the culture medium of step (b2) is below 5 ng/ml.

In a particular embodiment, a method is provided for the production of HECs, comprising step (a) culturing a suspension of PSCs thereby providing a plurality of first cell aggregates comprising said PSCs; and; step (b) comprising step (b1) of culturing the first cell aggregates obtained in step (a) in the presence of a SMAD agonist in particular BMP4, VEGF and bFGF, and, subsequent step (b2) of culturing the cells obtained in step (b1) in the presence at least during part of step (b2) of each of VEGF, bFGF, CHIR99021 and SB431542, wherein the culture medium during step (b2) does not comprise a SMAD pathway agonist (i.e. no SMAD pathway agonist is added to the culture medium for use in said step (b2)), and, wherein the culture medium is at least 80% replaced by fresh medium between step (b1) and step (b2), to generate a plurality of second cell aggregates comprising HECs. In a further embodiment, step (b1) lasts 2 days. In a further embodiment, step (b2) lasts 4 days. In a further embodiment, the culture medium is refreshed daily for at least 80% during step (b2) and VEGF, bFGF, CHIR99021 and SB431542 are administered to the cell culture as follows: day 1-VEGF, bFGF, CHIR99021 are added to the culture medium (thereby thus removing the BMP4 containing medium from the cell culture), day 2-VEGF, bFGF, CHIR99021 and SB431542 are added to the culture medium, day 3-VEGF, bFGF and SB431542 are added to the culture medium, and, day 4-VEGF and bFGF are added to the culture medium. In a further embodiment, VEGF, bFGF and BMP4 are dosed at 25 ng/ml, and, CHIR99021 and SB431542 are dosed at 3 μM in the cell culture during step (b). In a further embodiment, the second cell aggregates resulting from step (b) are subjected to step (c) as described herein to obtain HSCs, in particular using the defined HSC differentiation media as described herein. It is understood that the second cell aggregates are progressed to step (c) without disruption of the second cell aggregates such as for performing a cell selection or cell separation step.

In an embodiment the method of the invention further comprises step (c) culturing the plurality of second cell aggregates in culture medium to induce differentiation of the HECs comprised in the plurality of second cell aggregates to generate a plurality of third cell aggregates producing hematopoietic stem cells (HSCs) and allowing the HSCs to release from the plurality of third cell aggregates in the culture medium to obtain a population of human HSCs. It is understood that the second cell aggregates are progressed to step (c) without disruption of the second cell aggregates for performing a cell selection or separation step. The second cell aggregates remain intact and are not subjected to any type of purposive cell dissociation methodology (be it physical disruption, enzymatic or otherwise chemical) to break up at least in part the second cell aggregates.

In step (c) the plurality of second cell aggregates are cultured in culture medium to induce differentiation of the HECs comprised in the plurality of second cell aggregates to generate a plurality of third cell aggregates producing hematopoietic stem cells (HSCs) and allowing the HSCs to release from the plurality of third cell aggregates in the culture medium to obtain a population of HSCs thereby forming a single cell population comprising HSCs in suspension in the culture medium. During step (c) the HECs in the second cell aggregates are differentiated to at least in part become HSCs. It is understood and preferred that the HSCs release from the aggregates through budding. Budding is a process for releasing cells from a dense population of cells, such as a cluster of cells, e.g. cell aggregates, that is well-described in the art.

In some embodiments the method in accordance to the invention does not comprise a cell-selection or cell-separation step, in particular prior the formation of HSCs. Preferably, single cells or single HSCs, originate from the cell aggregates released from the cell aggregates (e.g. through budding) only. This means no forces, mechanical or other, are applied to dissociate cells present in the first, second or third cell aggregates described herein. Nor are specified (sub-) populations of cells present in the culture medium selectively harvested, removed or separated from the other cells present in the culture medium or culture vessel, e.g. based on their marker profile.

In some embodiments, step (c) comprises a step (c1) and a step (c2), wherein step (c2) comprises that the single cell population comprising the HSCs (which spontaneously budded from the third cell aggregates) are separated from the remaining cell aggregates. It is contemplated that said HSCs are separated from said remaining cell aggregates by using methods commonly used in the art, such as but not limited to filtration, centrifugation or elutriation (e.g., counterflow centrifugal elutriation), for example by using a Gibco™ CTS™ Rotea™ Counterflow Centrifugation System. In one preferred embodiment the separation is by counterflow centrifugal elutriation. Preferably, step (c1) comprises the culturing of the plurality of second cell aggregates in culture medium to induce differentiation of the HECs comprised in the plurality of second cell aggregates to generate a plurality of third cell aggregates producing hematopoietic stem cells (HSCs) and allowing the HSCs to release from the plurality of third cell aggregates in the culture medium to obtain a population of HSCs thereby forming a single cell population of comprising the HSCs in suspension in the culture medium.

Suitable culture conditions for differentiating HECs to HSCs are known to the skilled person. For example, IMDM medium can be used. The culture medium may optionally be supplemented with supplements selected from but not limited to BSA (e.g. between 0.04% and 1%, preferably about 0.2%), ITS-X (e.g. between 0.2% and 5%, preferably about 1%), β-me (e.g. between 11 and 275 μM, preferably about 55 μM), Ascorbic acid-2P (e.g. between 10 and 250 μg/mL, preferably about 50 μg/mL), and GlutaMax (e.g. between 0.2% and 5%, preferably about 1%). Growth factors to induce and/or expand HSC for use in the present method may be selected from the group comprising TPO (e.g. between 10 and 250 ng/ml, preferably about 50 ng/mL), hSCF (e.g. between 10 and 250 ng/ml, preferably about 50 ng/ml), FIT3-L (e.g. between 10 and 250 ng/ml, preferably about 50 ng/ml), IL-6 (e.g. between 2 and 50 ng/ml, preferably about 10 ng/ml), and/or IL-3 (e.g. between 2 and 50 ng/ml, preferably about 10 ng/ml). Non-limiting examples of other suitable growth factors for step (c) are Wnt3a (e.g. between 1 and 500 ng/ml), bFGF (e.g. between 1 and 100 ng/mL, preferably about 10 ng/ml), FICZ (e.g. between 0.5 and 20 μM) or TCDD (e.g. between 1 nM and 1 μM). Non-limiting examples of other suitable growth media for step (c) are StemPro, Hams-F12, APEL and RPMI. Non limiting examples of suitable supplements are KO-SR, N-2 suppl, B-27 Suppl.

In some embodiments, the method in accordance to the invention comprises that the culture medium to induce differentiation of the HECs comprises bFGF. It is preferred that said culture medium to induce differentiation of the HECs comprises bFGF in combination with one or more further growth factor initiating HSC differentiation and/or expansion. For example, said culture medium may comprise bFGF in combination with FLT3L and/or SCF and one or more further growth factor initiating HSC differentiation and/or expansion. bFGF is added to the HSC differentiation medium in a concentration between 5 and 125 ng/ml, between 5 and 75 ng/ml, between 5 and 50 ng/ml. In particular embodiments, bFGF is present in the HSC differentiation medium at about 25 ng/ml or about 10 ng/ml.

In one preferred embodiment the method in accordance to the invention comprises that the culture medium to induce differentiation of the HECs comprises bFGF in combination with one or more further growth factor initiating HSC differentiation and/or expansion, such as in combination with FLT3L and one or more further growth factor initiating HSC differentiation and/or expansion, and/or, such as in combination with SCF and one or more further growth factor initiating HSC differentiation and/or expansion.

It is contemplated that the one or more further growth factor initiating HSC differentiation does not comprise one or more notch ligands. Therefore, in one embodiment the method in accordance to the invention, preferably the step (c), wherein a culture medium is provided to induce differentiation of the HECs, does not comprise the provision of a notch ligand to the culture medium and/or cells. More preferably, the HECs and/or HSCs obtained herein are not exposed to nor contacted with a notch ligand, e.g., Jagged1, Jagged2, Delta-like1 (DLL1), Delta-like3 (DLL3), or Delta-like4 (DLL4), during the method of the invention.

In some embodiments, when provided to a culture medium for differentiating HECs to HSCs in step (c) of the methods of the invention, IL-3 may be provided in a concentration between from 0.5 ng/mL and 50 ng/ml. In a particular embodiment IL-3 is present in the HSC differentiation medium at 1 ng/ml, in particular to provide HSC for further differentiation into lymphoid cells, such as NK-cells or T-cells (including Treg-cells). In an alternative particular embodiment IL-3 is present in the HSC differentiation medium at 10 ng/ml, in particular to provide HSC for further differentiation into myeloid cells.

In a particular embodiment, the culture medium to induce differentiation of the HECs in step (c), such as in step (c1), comprises growth factors TPO, hSCF, FIT3-L, IL-6 and IL-3. In a further embodiment, said hSCF, TPO and FIt3L are present at about 50 ng/ml, IL-3 and IL-6 are present at about 10 ng/ml.

In a particular embodiment, the culture medium to induce differentiation of the HECs, for example in step (c), such as in step (c1), comprises growth factors hSCF, FIT3-L and bFGF. In a further embodiment said hSCF and FIt3L are present at about 50 ng/ml, and bFGF is present at about 10 ng/ml.

In another particular embodiment, the culture medium to induce differentiation of the HECs in step (c), such as in step (c1), comprises growth factors TPO, hSCF, FIT3-L, IL-6, IL-3 and bFGF. In a further embodiment said hSCF, TPO and FIt3L are present at about 50 ng/ml, bFGF and IL-6 are present at about 10 ng/ml, and, IL-3 is present at a concentration between and including 1 and 10 ng/ml.

Particular culture media compositions for HEC and/or HSC differentiation described herein, are also considered part of the invention.

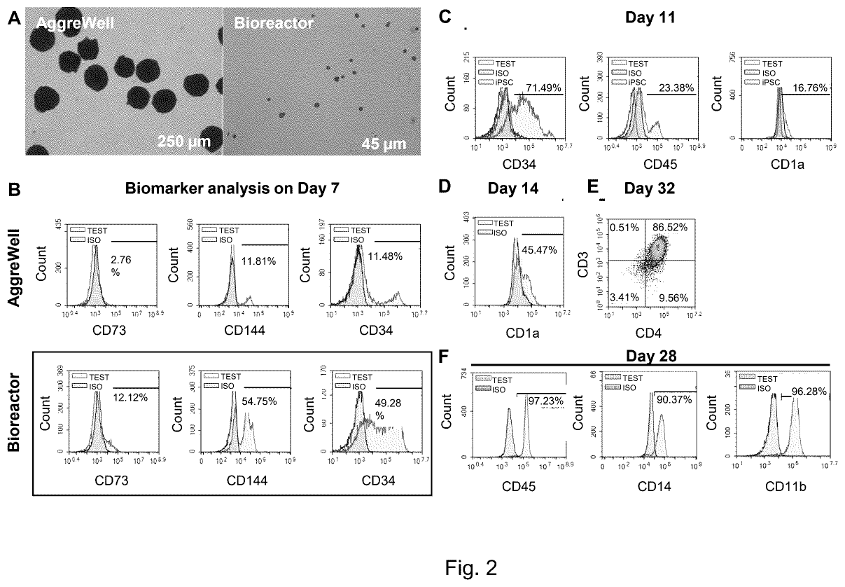

Alternatively, instead of using a culture medium for HSC differentiation in step (c), it is further envisioned that the medium used in step (c) may be a medium for further differentiation towards hematopoietic lineages. Non limiting examples are differentiation towards lymphoid or myeloid cell lineages. Without wishing to be bound by theory, the inventors believe that by directly differentiating aggregates comprising HECs to e.g., the lymphoid or myeloid lineages an efficient differentiation can be achieved. It is further theorized that when HECs are cultured in lineages specific culture medium (e.g., lymphoid or myeloid lineage specific culture medium), differentiation through HSCs still occurs, meaning that the aggregates still produce HSCs that are released from the aggregates and further differentiate towards lymphoid or myeloid cells. This further supported by the data represented in FIGS. 2C-2F, where HECs were directly differentiated towards T cells (lymphoid; FIG. 2C-E) or monocytes (myeloid; FIG. 2F). The lymphoid cells can be seen to express HEC/HSC markers at day 11 (CD34, FIG. 2C), T cell marker progenitor markers at day 14 (CD1a, FIG. 2D) and T cell markers at day 32 (CD3 and CD4, FIG. 2E). The myeloid cells can be seen to express HSC markers (CD45) and monocyte markers (CD14, CD1b; FIG. 2F) at day 28.

Therefore in an embodiment step (c) the plurality of second cell aggregates are cultured in culture medium to induce differentiation of the HECs comprised in the plurality of second cell aggregates to generate a plurality of third cell aggregates producing hematopoietic stem cells (HSCs) and allowing the HSCs to release from the plurality of third cell aggregates in the culture medium to obtain a population of human HSCs, wherein the culture medium in step (c) is for differentiation to lymphoid cells or myeloid cells. Culture conditions that lead to either lymphoid or myeloid lineage differentiation are known to the skilled person. For example for myeloid differentiation exemplary differentiation media that can be used from day 6 onwards are: medium 1: XVIVO15+MCSF 100 ng/ml+IL3 25 ng/ml; or medium 2: XVIVO15+TPO 50 ng/ml +Flt3L 10 ng/ml+huSCF 50 ng/ml+MCSF 80 ng/ml+GMCSF 10 ng/ml. For lymphoid/T-cell differentiation an exemplary differentiation medium that can be used from day 6 onwards is: HEC aggregates plated on vitronectin coated tissue culture plastic in XVIVO15 medium+VEGF 50 ng/ml+huSCF 100 ng/ml+bFGF 10 ng/ml+IL7 20 ng/ml).

Thus in step (c) it is possible to focus on HSC differentiation to maximize differentiation and amplification of HSCs, which can either be used directly or cryopreserved for later use and differentiation towards desired cell types. Alternatively, the HECs may be pushed to differentiate directly towards a specific lineage (e.g. lymphoid or myeloid) immediately, thereby reducing process steps, simplifying the process and reducing the length of the overall manufacturing process.

In an embodiment step (c) is performed for at least about 4 days, such as for example at least 3, 4, 5, 6, 7, or 8 days, and/or at most about 14 days, such as for example at most 14, 13, 12, 11, 10, 9, or 8 days. Preferably, step (c) is be performed between 3 and 14 days, for example between 4 and 12, between 5 and 10, or between 6 and 8 days. Alternatively, step (c) is to be performed between 5 and 14, between 8 and 14, between 10 and 14, or between 12 and 14 days. It was surprisingly found that with the method of the invention it has become possible to allow the aggregates to produce HSCs for a prolonged period of time. The HSCs thus obtained may be further expanded, for example after cryopreservation and thawing of the cells, for example as discussed herein. The cells may be expanded in the same or in a different (bio) reactor.

In an embodiment at least 60-90% (such as at least 60, at least 70, at least 80 or at least 90%) of the cells released from the plurality of third cells aggregates are HSCs.

In some embodiments the single cell population separated from the remaining cell aggregates comprising the HSCs comprises at least 50%, at least 60%, at least 70%, at least 75%, at least 80% HSC cells, or at least 90% HSC, wherein the HSC cells express the marker(s) CD34, CD43 and/or CD45.

In an embodiment the culture medium during step (c) is replaced every day, every other day, every 3rd day, every 4th day, or combinations thereof (e.g., the first replacement is after 2 days, and a subsequent replacement of the culture medium is after 4 days). In a particular embodiment, the culture medium is refreshed more frequently in the beginning of step (c). In a particular preferred embodiment, the culture medium during step c) is replaced or refreshed every day.

In some embodiments, during step (c) all medium is replaced or refreshed. However, it is surprisingly found that it is possible to design robust and reproducible manufacturing processes yielding well-differentiated cells in high quantities in closed culture systems by not collecting all of the culture medium from the culture vessel and replacing it with fresh culture medium, but by collecting only part of the culture medium from the culture vessel before a subsequent, fresh, culture medium (and, where required, growth factors such as those disclosed herein) is added to the remaining culture medium in the culture vessel (and thus wherein part of the previous medium is mixed with the subsequent medium). For example, in one embodiment at most 95 vol. %, 90 vol. %, 85 vol. %, or 80 vol. % of the culture medium in the culture vessel is collected to enable efficient switching of media composition and/or harvesting of (single) cells, e.g., HSC cells, or material secreted by the cells into the culture medium. For example, in this embodiment, in case the culture vessel comprises 10 liters of culture medium, at most 9500, 9000, 8500 or 8000 milliliter of the culture medium is collected from the culture vessel (and from which HSC may be isolated and obtained).

At the same time, it was found that, preferably, at least 30 vol. %, 40 vol. % 50 vol. %, 60 vol. % or 70 vol. % of the culture medium is collected from the culture vessel. Therefore, in some embodiments between, for example between 30 vol. %-95 vol. %, or between 40 vol. % and 95 vol. % or between 50-95 vol. %, or between 60-90 vol. % or between 60-80 vol. %, or between 70-90 vol. % or between 70-80 vol. % of the culture medium is collected from the culture vessel and, preferably, replaced or refreshed with new culture medium. As will be understood by the skilled person, the amount or percentage of medium collected may vary between different moments of collection medium according to the method of the invention. For example, during a first medium collection during step (c) 70 vol % of the culture medium in the culture vessel may be collected and replaced, whereas during a subsequent medium collection during step (c), the same, more or less (e.g. 50 vol. % or 75 vol. %) may be collected and replaced. In some embodiments growth factors are added directly provided to culture medium already present in the culture vessel or closed culture system, without the substantial replacement of old culture medium or the substantial addition of new culture medium.

The markers used or described herein are generally known to the person skilled in the field of stem cell differentiation. For easy reference the following tables are provided:

| Marker | Also known as | Encoding gene (human) |
|---|---|---|
| CD34 | | ENSG00000174059 |
| CD73 | NT5E | ENSG00000135318 |
| CD144 | CDH5 | ENSG00000179776 |
| CD45 | PTPRC | ENSG00000081237 |
| OCT4 | POU5F1 | ENSG00000230336 |
| SOX2 | | ENSG00000181449 |
| NANOG | | ENSG00000111704 |
| CD43 | SPN | ENSG00000197471 |
| CD1a | | ENSG00000158477 |
| CD7 | | ENSG00000173762 |
| CD4 | | ENSG00000010610 |
| CD117 | KIT | ENSG00000157404 |
| CD49f | ITGA6 | ENSG00000091409 |
| CD90 | THY1 | ENSG00000154096 |
| CD44 | | ENSG00000026508 |
| CD14 | | ENSG00000170458 |
| CD1b | | ENSG00000158485 |

| Marker | Expression profile |
|---|---|
| CD34 | HECs and HSCs are CD34 positive |
| CD73 | HECs are CD73 negative |
| CD144 | HECs are CD144 positive |
| CD45 | HSCs are CD45 positive |
| OCT4 | PSCs are OCT4 positive |
| SOX2 | PSCs are SOX2 positive |
| NANOG | PSCs are NANOG positive |
| CD43 | HSCs are CD43 positive |
| CD1a | proTcells (T-cell progenitors) are CD1a positive |
| CD7 | Thymocytes/mature T cells are CD7 positive |
| CD4 | T helper cells, monocytes, macrophages, and |
| | dendritic cells are CD4 positive |
| CD117 | HSCs are CD117 positive |
| CD49f | LT-HSC are CD49f positive |
| CD90 | HSCs are CD90 positive |
| CD44 | T cells are CD44 positive |
| CD14 | Macrophages are CD14 positive |
| CD1b | Dendritic cells, monocytes and thymocytes are CD1b positive |

In an embodiment the plurality of first cell aggregates have an average size of about 20-55 micrometers or have an average size of about 25-50 micrometers or have an average size of about 30-40 micrometers and the plurality of second cell aggregates have an average size of about 35-200 micrometers in diameter. In a further embodiment, the plurality of first cell aggregates have an average size of about 49, 48, 47, 46, 45 or less micrometer in diameter.

It is appreciated that while differentiating, the cells in the plurality of first and second cell aggregates also proliferate, meaning that the cell aggregates grow in size over time. Therefore, in an embodiment the plurality of second cell aggregates, for example 3-8 days after step a) e.g. preferably 5-6 days after step a) e.g. on day 6 have an average size of about 150-600 micrometers in diameter. For example the aggregates may have an average size of at least 150, 160, 170, 180, 190, or even 200 micrometers, and/or have an average size of 600, 580, 560, 540, 520, or 500 micrometers in diameter or less. Therefore, the average size of the plurality of second cell aggregates has preferably been increased 10 to 20 fold, e.g. 10, 11, 12, 13, 14, 15, 16, 17, 18, 19, or 20 fold, when compared to the average size of the plurality of first cell aggregates.

In some embodiments, the plurality of second cell aggregates have an average size of about 150 to 600 μm in diameter.

In an embodiment the HECs that are comprised in the plurality of second cell aggregates express the marker(s) CD34 and/or CD144; and/or the plurality of second cell aggregates comprises at least 30% cells expressing CD34; at least 30% cells expressing CD144; and/or at most 30% cells expressing CD73. It is further theorized that absence of the marker CD235a may be used, therefore in an alternative embodiment the HECs that are comprised in the plurality of second cell aggregates express the marker(s) CD34 and/or CD144; and/or the plurality of second cell aggregates comprises at least 30% cells expressing CD34; at least 30% cells expressing CD144; and/or at most 30% cells expressing CD73; and/or, preferably at most 30% cells expressing CD235a. It is considered that even when a cell is considered to not express a marker, the marker may still be present in low amounts on the cell. Therefore when referring to a marker, the terms “expressed” or “not expressed” indicate whether the marker is present above or below a certain threshold. The threshold for the respective marker can easily be determined by the skilled person by calibrating using reference samples, as the skilled person knows and understands. Preferably the cells that are comprised in the plurality of second cell aggregates comprise at least 30% cells expressing CD34, for example 30%, 35%, 40%, 45%, 50% or more cells expressing CD34, and/or comprises at least 30% cells expressing CD144, for example 30%, 35%, 40%, 45%, 50% or more cells expressing CD144, and/or comprises at most 30% cells expressing CD73, for example 25%, 20%, 15%, 10%, 5%, 2% or less cells expressing CD73. Alternatively, the cells that are comprised in the plurality of second cell aggregates comprise at most 30% cells expressing CD235a, for example 25%, 20%, 15%, 10%, 5%, 2% or less cells expressing CD235a.

In an embodiment the human HSCs released from the plurality of third cell aggregates, preferably wherein the human HSCs are in the form of a suspension of single cells, express the marker(s) CD34, CD43 and/or CD45. Preferably the population of human HSCs obtained in step (c) (or in step (c1) and/or (c2)) comprises at least 50% cells expressing CD34; at least 50% cells expressing CD43; at least 50% cells expressing CD45; and/or at most 30% cells expressing CD14. Preferably at least 50% of the cells, or more, express the express the marker(s) CD34, CD43 and CD45. Preferably the human HSCs in the form of a suspension of single cells and/or the population of human HSCs obtained in step (c) comprises at least 50% cells expressing CD34, for example 50%, 55%, 60%, 65%, 70%, 75%, or more cells expressing CD34, and/or comprises at least 50% cells expressing CD43, for example 50%, 55%, 60%, 65%, 70%, 75%, or more cells expressing CD43, and/or comprises at least 50% cells expressing CD45, for example 50%, 55%, 60%, 65%, 70%, 75%, or more cells expressing CD45. Preferably the human HSCs in the form of a suspension of single cells and/or the population of human HSCs obtained in step (c) comprises at most 30% cells expressing CD14, for example 30%, 25%, 20%, 15%, 10% or less cells expressing CD14.

In an embodiment the population of human HSCs are in the form of a suspension of single cells comprising at least 50% HSCs, or at least 75% HSCs, or at least 90% HSC, such as for example 50%, 60%, 70%, 80%, 90% or more HSC cells, preferably wherein the HSC cells express the marker(s) CD34, CD43 and/or CD45.

In an embodiment, culturing the plurality of first cell aggregates in step (b) wherein step (b) comprises a step (b1) of culturing wherein the culture medium does comprise a SMAD pathway agonist, preferably BMP4, and, a step (b2) of culturing wherein the culture medium is at least partly replaced by fresh medium which does not comprise a SMAD pathway agonist, and wherein step (b1) is before step (b2).

In an embodiment the human HSCs released from the plurality of third cell aggregates are cryopreserved to obtain cryopreserved HSCs. The skilled person is aware of suitable protocols for cryopreservation. For example, Cryostor® CS10 Cryomed™ medium may be used.

In an embodiment the method comprises a step (d) that comprises further culturing the human HSCs and/or the cryopreserved HSCs in culture medium to induce proliferation thereof. It is understood that the HSCs can be isolated from the culture and either be used directly or be cryopreserved. For example, the HSCs may be matured in a suitable medium. Suitable media are known to the skilled person, for example, IMDM medium may be used, which may for example be supplemented with but not limited to BSA (e.g. between 0.04% and 1%, preferably about 0.2%), ITS-X (e.g. between 0.2% and 5%, preferably about 1%), β-me (e.g. between 11 and 275 μM, preferably about 55 μM), Ascorbic acid-2P (e.g. between 10 and 50 g/mL, preferably about 50 μg/mL), Glutamax (e.g. between 0.2% and 5%, preferably about 1%), TPO (e.g. between 20 and 500 ng/ml, preferably about 100 ng/ml), hSCF (e.g. between 10 and 250 ng/ml, preferably about 50 ng/ml), FIT3-L (e.g. between 10 and 250 ng/ml, preferably about 50 ng/ml), IL-6 (e.g. between 2 and 50 ng/ml, preferably about 10 ng/ml), IL-3 (e.g. between 2 and 50 ng/mL), UM729 (e.g. between 100 and 2500 nM, preferably about 500 nM to 1000 nM), and/or Scriptaid (e.g. between 0.2 and 5 μM, preferably about 1 μM). It was also found that a medium comprising UM729, preferably in a concentration as provided herein, preferably a medium for maturing and/or expanding the HSCs obtained by the method of the invention, allows for obtaining of a high quality, i.e. maintaining their biomarker profile as present in the cell population resulting from step (c) described herein, matured and/or expanded HSCs.

The further addition of IL-3 is contemplated to further increase the yield/expansion ratio of HSCs obtained by the methods described herein, in particular when downstream differentiation of the HSC to myeloid is envisaged.

In an embodiment the expansion ratio PSC: HSC is at least 50, at least 100, or at least 400, for example after expansion of the HSCs obtained with the method of the invention. For example, during steps (a) and (b) of the method the expansion ratio PSC: HSC is at least 10, or even 20, 30, 40, 50, 60, 70, or 80 or more. Additionally in optional step (d) of the method the expansion of HSCs is preferably at least 2, 3, 4, 5, 6, 7, 8, 9, or 10 or more. When used herein the expansion of HSCs refers to (number of HSCs at the end of step (d)): (number of HSCs at the start of step (d)). Therefore, in an embodiment the expansion of HSCs in step (d) is 2 or more, preferably 4 or more most preferably 10 or more. When used herein the expansion ratio of PSC: HSC refers to the amount of cells generated (in this case HSCs), compared to the amount of cells provided in the culture in step (a) (in this case PSCs). For example, when 10.000 PSCs are provided and 550.000 HSCs are generated/obtained the expansion ratio is more than 50, i.e. 55.

It is understood that UM729 is a Pyrimidoindole derivative, the compound is previously described as an agonist of hematopoietic stem cell self-renewal (Fares et al 2014 Science 345, 1509-1512). Other agonists with a similar effect are UM171.

In an embodiment the human HSCs are further cultured to induce differentiation of the cells into common erythroid/megakaryocytic progenitor cells, erythrocytes, megakaryocytes, platelets, common lymphoid progenitor cells, lymphoid lineage cells, lymphocytes, T lymphocytes, natural killer (NK) cells, common myeloid progenitor cells, myeloid-derived suppressor cells, common granulomonocytic progenitor cells, monocytes, macrophages, and/or dendritic cells. Non-limiting examples of T lymphocytes are helper T cells, cytotoxic T cells, memory T cells, regulatory T cells, natural killer T cells, mucosal associated invariant T cells and gamma-delta T cells. A regulatory T cell may also be referred to as a Treg cell, and may be a resting Treg cell, an activated Treg cell or an immunosuppressive Treg cell. Induction of these cells may be performed using methods known to the skilled person. In particular embodiments, the HECs and/or HSCs are further cultured to induce differentiation of the cells into lymphoid lineage cells, such as lymphoid progenitor cells, T-cells (including alpha/beta T-cells and gamma/delta T cells), helper T (TH) cells, regulatory T (Treg) cells, natural killer (NK) cells. In alternative embodiments, the HECs and/or HSCs are further cultured to induce differentiation of the cells into myeloid cells. Alternatively, the HECs and/or HSCs are further cultured to induce differentiation of the cells into myeloid cells.

In an embodiment the cell culture is performed in a bioreactor.

There are many types of closed culture systems, culture vessels or bioreactors that are suitable for the method as disclosed herein. In particular, the bioreactor as provided herein preferably contains a stirrer mechanism or other means that allow for mixing of the culture medium comprising the cells during cultivation. Examples of suitable systems are custom stainless steel/glass reactors or single use systems such as Ambr and Biostat (Sartorius), PBS (PBS biotech), DASbox and Bioflo (Eppendorf), Appiflex (Applikon), Wave and Xuri (GE/Cytiva) in a typical volume range of a few milliliters up to hundreds of liters. Further particular examples of closed culture systems for performing the methods provided herein are described in WO2022019768.

Further, the bioreactor as disclosed herein may comprise controls for oxygen and CO₂ and probes for measuring the biomass, cell density, pH-value of the culture medium, lactate concentration, and/or for measuring the amount of dissolved oxygen contained in the culture medium, as well as introduced in the culture vessel. Such probes and controls are known to the skilled person.

It is preferred that the method, preferably when performed in a closed system or (closed) bioreactor that the method does not comprise a step comprising selection of cells (i.e. a cell selection step) and/or enrichment for a specific cell-type (i.e. specific cell-type enrichment step). In some embodiments no single cell sorting is performed on the herein obtained HECs and/or HSCs and/or any other of the cells obtained by further culturing and/or differentiating HSCs.

An advantage of the performing of the method of the invention in such a closed system or (closed) bioreactor, preferably without said cell selection and/or cell-type enrichment step is that, the method of the invention allows for a method that does not require that the system and/or bioreactor is opened (for example by opening a lid or valve), e.g. thereby exposing the cells to air, contaminations and/or a method step performed by a human etc., in order to perform a step to manipulate the cells.

Another advantage of the method in accordance to the invention, preferably when performed in a closed system or (closed) bioreactor is that it allows for obtaining cells and/or cell aggregates having a high yield (e.g. an expansion rate of PSC: HSC of at least 50, at least 100, or at least 400) and/or high purity (e.g. expressing levels of CD144, CD45, CD43 and/or CD34 markers as described herein). Therefore, it is contemplated that cells obtained by the method as broadly described herein are suitable for therapeutic use, e.g. use in the treatment of diseases and/or disorders, or at least amenable for differentiation into cell for therapeutic use.

It is a further advantage of the methods provided herein that the resulting composition comprising the desired cells such as a cell culture comprising HECs or HSCs of the method in accordance to the invention, e.g. the cell culture of PSCs, HECs and/or HSCs, does not comprise (exogenous) feeder cells and/or a solid support (e.g. in the form of nanobeads). Such feeder cells and/or solid support are well known by the skilled person and commonly used in the culturing of cells. When contemplating therapeutic use of the cells manufactured using the methods described herein, the absence of feeder cells and/or solid support, such as but not limited to microbeads, is considered advantageous by avoidance of potential sources of contamination of the final therapeutic product.

In a second aspect the invention relates to cells, or to a composition (of cells), comprising a first population and a second population, wherein the first population is in the form of cell aggregates comprising HEC cells and wherein the second population is in the form of a suspension of single cells comprising HSC cells, preferably wherein the second population comprises at least 50% HSC cells, or at least 75% HSC cells, or at least 90% HSC, wherein the HSC cells express the marker(s) CD34, CD43 and/or CD45, or wherein the cells comprise of the second population. Therefore the second population comprises at least 50% cells expressing CD34, for example 50%, 55%, 60%, 65%, 70%, 75%, 80%, 85%, 90% or more cells expressing CD34, and/or at least 50% cells expressing CD43, for example 50%, 55%, 60%, 65%, 70%, 75%, 80%, 85%, 90% or more cells expressing CD43, and/or at least 50% cells expressing CD45, for example 50%, 55%, 60%, 65%, 70%, 75%, 80%, 85%, 90% or more cells expressing CD45.

In some embodiments, there is provided for a population of HECs and/or HSCs and/or of differentiated cells selected from the group consisting of lymphoid cells, T-cells (including alpha/beta T-cells and gamma/delta T cells), helper T (TH) cells, regulatory T (Treg) cells and natural killer (NK) cells obtainable by the method as provided herein.

In an embodiment the first, second, and/or third culture medium is in a volume of at least 1 Liter, more preferably at least 10 Liter, more preferably at least 50L, more preferably at least 100L.

In a third aspect the invention relates to a population of human HSCs or cryopreserved HSCs as defined herein.

In an embodiment the population of HSCs and/or human cryopreserved HSCs comprises at least 70% cells expressing CD34; at least 70% cells expressing CD43; and/or at least 70% cells expressing CD45, and wherein the percentage of cells expressing CD34, CD43 and CD45 remains stable when the cells are proliferated for one or two days according to step (d) in a suitable culture medium at a density of 100,000-1,000,000 million cells per mL, more preferential 333,333 viable cells/ml. For the purpose of the invention, the percentage of cells expressing CD34, CD43 and CD45 is deemed stable when that percentage of cells is at most reduced by 15%. For example, IMDM medium may be used, which may for example be supplemented with but not limited to BSA (e.g. between 0.04% and 1%, preferably about 0.2%), ITS-X (e.g. between 0.2% and 5%, preferably about 1%), β-me (e.g. between 11 and 275 μM, preferably about 55 μM), Ascorbic acid-2P (e.g. between 10 and 50 μg/mL, preferably about 50 μg/mL), Glutamax (e.g. between 0.2% and 5%, preferably about 1%), TPO (e.g. between 20 and 500 ng/ml, preferably about 100 ng/mL), hSCF (e.g. between 10 and 250 ng/ml, preferably about 50 ng/ml), FIT3-L (e.g. between 10 and 250 ng/ml, preferably about 50 ng/ml), IL-6 (e.g. between 2 and 50 ng/ml, preferably about 10 ng/ml), IL-3 (e.g. between 2 and 50 ng/ml), UM729 (e.g. between 100 and 2500 nM, preferably about 500 nM), and/or Scriptaid (e.g. between 0.2 and 5 μM, preferably about 1 μM),

For example, the population of cells comprises at least 70% cells expressing CD34, for example 70%, 75%, 80%, 85%, or 90%, and/or the population of cells comprises at least 70% cells expressing CD43, for example 70%, 75%, 80%, 85%, or 90%, and/or the population of cells comprises at least 70% cells expressing CD45, for example 70%, 75%, 80%, 85%, or 90%.

In a fourth aspect the invention relates to a culture medium or a bioreactor with culture medium comprising the cells or population of cells as described herein, or comprising cell or aggregates as defined herein.

In some embodiments, the invention relates to a culture system or a bioreactor, preferably a closed culture system or closed bioreactor, with culture medium comprising the cells or population of cells as provided herein or comprising cells or cell aggregates as provided herein.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein.

All references cited herein, including journal articles or abstracts, published or corresponding patent applications, patents, or any other references, are entirely incorporated by reference herein, including all data, tables, figures, and text presented in the cited references. Additionally, the entire contents of the references cited within the references cited herein are also entirely incorporated by references.

It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

It will be understood that all details, embodiments and preferences discussed with respect to one aspect of embodiment of the invention is likewise applicable to any other aspect or embodiment of the invention and that there is therefore not need to detail all such details, embodiments and preferences for all aspect separately.

Having now generally described the invention, the same will be more readily understood through reference to the following examples which is provided by way of illustration and is not intended to be limiting of the present invention. Further aspects and embodiments will be apparent to those skilled in the art.

### EXAMPLES

### Example 1

### Results

### Aggregate Size, During Differentiation Day 0 to 2

Following 3-week preculture, PSCs were inoculated to form aggregates either in AggreWellTM800 plates or DASbox stirred tank bioreactor systems with a pitch-blade. 2-3 days following inoculation, AggreWellTM800 plates generated aggregates with an average diameter of 250 μm, while bioreactors generated aggregates of 45 μm (FIG. 2A). Subsequent parallel cultures of aggregates with different sizes indicated that smaller aggregates differentiated with higher efficiency to populations positive for HEC markers. Briefly, at 7 days after formation (corresponding to 6 days of culture in HEC differentiation medium), populations were 49.28% positive for CD34, 54.75% positive for CD144, and had low expression (12.12%) for CD73—a marker for HEC precursors (FIG. 2B). In contrast, larger aggregates expressed CD34 and CD144 at only 11.48% and 11.81%, respectively. At the day 6 timepoint, aggregates from this condition had a size of 360+36 μm and density of 867,683 cells per ml, representing an approximately 40-fold expansion between day 0 to day 6.

When cultured in T-lymphocyte-inducing conditions, small aggregates progressed to successfully form populations positive for the hematopoietic cell marker CD45 (23.38%; FIG. 2C), while still expressing only 16.76% of CD1a, a marker indicative of further differentiation towards the lymphoid lineage. Further culture led to lymphoid precursors (45.47% CD1a+; FIG. 2D) and then towards either CD3/CD4 positive T cell populations (48.61%; FIG. 2E). This indicates that HECs are capable of forming white blood cell derivatives of the lymphoid lineage. In addition, culture of day-7 HEC in conditions favoring monocyte differentiation resulted in over 90% CD14+, CD11b+, or CD45+ populations (FIG. 2F), demonstrating the capability of forming white blood cells of the myeloid lineage.

### Aggregate Density on Differentiation Day 0

Following initial formation, day 0 aggregates were reseeded at different densities, to determine the optimal conditions for HEC induction. High seeding densities of approximately 33,333 aggregates per ml resulted in formation of large clusters, likely due to aggregate fusion (FIG. 3A). Under these conditions, HEC differentiation was poor, as evidenced by CD34 and CD144 expression at 3.54% and 5.29%, respectively, in day 7 aggregates (FIG. 3B). In contrast, low seeding densities of approximately 667 aggregates per ml allowed expression of CD34 at 49.28% and CD144 at 54.75%, with CD73 expressed at only 12.12% (FIGS. 2B and 3B). Seeding densities of approximately 6,667 aggregates/ml generated intermediate results. Importantly, when cultured in HSC-inducing conditions, the 667 aggregate/ml seeding density also allowed upregulation of the hematopoietic cell marker CD45 in the highest % of the population (69.95%), compared to other conditions tested (43.11% and 7.08% for 6,667 and 33,333 aggregates/ml).

### Medium Composition for HSC Generation, on Differentiation Day 0 to Day 6

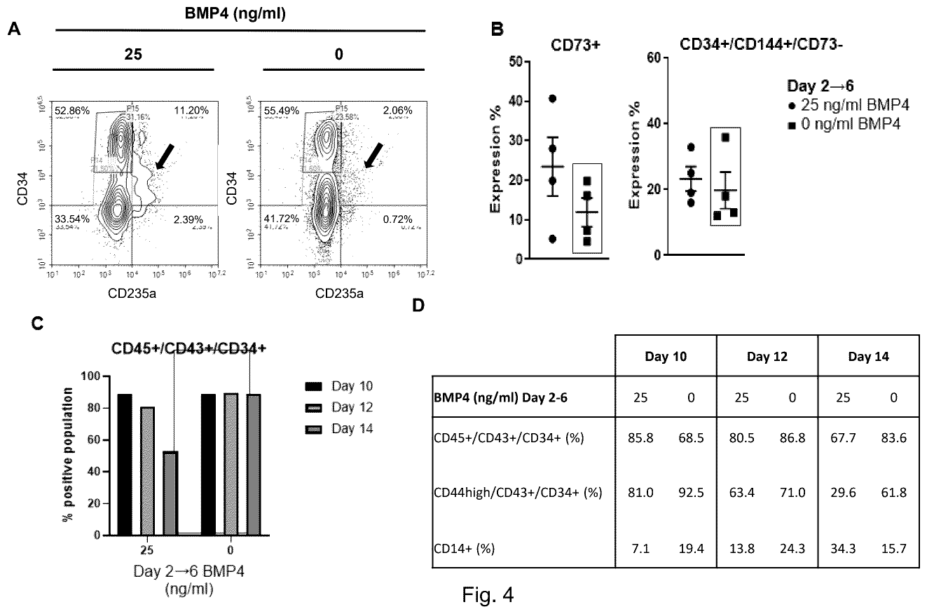

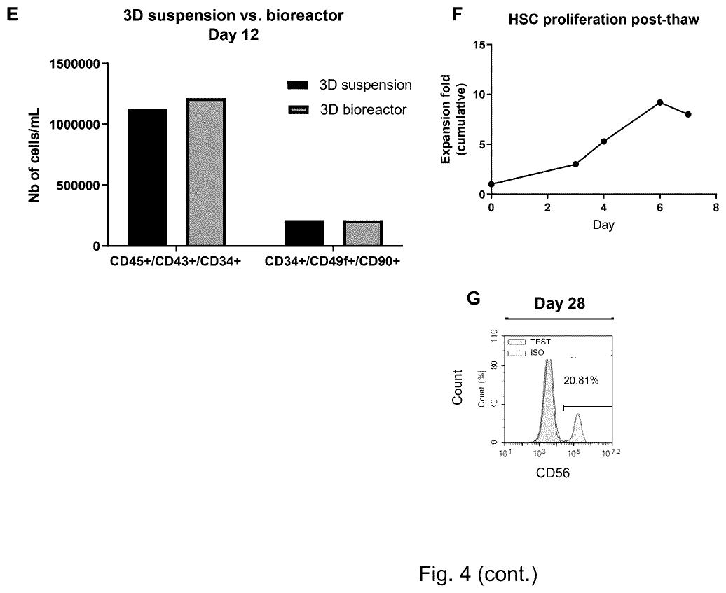

Further refinement of the HSC differentiation process was sought by testing different media formulations between days 0 and 6 of differentiation. Omission of BMP4 supplement during medium changes performed between differentiation day 2 to day 6 generated HSC with a more stable identity. Specifically, this medium formulation reduced unwanted cell populations with the primitive hematopoiesis marker CD235a from approximately 11.2% to 2.06% (FIG. 4A) as well as formation of unwanted CD73+ cell populations on differentiation day 6 (FIG. 4B). At the same timepoint, BMP4 omission did not affect the efficiency of HEC formation, evidenced by the unchanged CD34+/CD144+/CD7-population (FIG. 4B). Varying BMP4 concentration from 25 ng/ml to 10 ng/ml or 100 ng/ml between day 0 to day 6 increased CD34+/CD144+/CD7-populations by approximately 10%, but also increased variability between experiments as well as presence of unwanted CD235a populations.

At later differentiation stages, and following culture in HSC-inducing media, BMP4 omission enabled formation of cell populations that maintained a high purity (>80%) of CD45+/CD43+/CD34+markers at least between differentiation day 10 and 14 (FIG. 4C). In contrast, treatment with 25 ng/ml BMP4 between day 0 to day 6 led to a peak of HSC identity on day 10, which declined from 88.95% to 52.85% by day 14. Together with an overall increase in CD44high/CD43+/CD34+ and decrease in CD14+ populations (FIG. 4D), day 2 to day 6 BMP4 omission improved the efficiency and stability of PSC differentiation to HSC. The efficiency and yield of PSC differentiation to HSC was comparably high when performed in small-scale 3D suspension cultures with an orbital shaker or larger-scale bioreactors (1,172,000 CD45+/CD43+/CD34+ cells/ml; FIG. 4E), demonstrating the robustness and scalability of the system.

HSC were cryopreserved using CryoStor CS10 freezing medium and thawed successfully, maintaining a 90% post-thaw viability and over 80% CD45+/CD43+/CD34+identity for at least 3 days. Under culture in HSC proliferation media, the number of cells between days 0 to 7 post-thaw increased by approximately 10-fold (FIG. 4F), indicating that HSCs had a late-stage, self-renewing identity.

### Example 2—Impact of First Aggregate Size

### Methods

Aggregate Size of First Aggregates of D-2 to 0 vs. D-3-D0

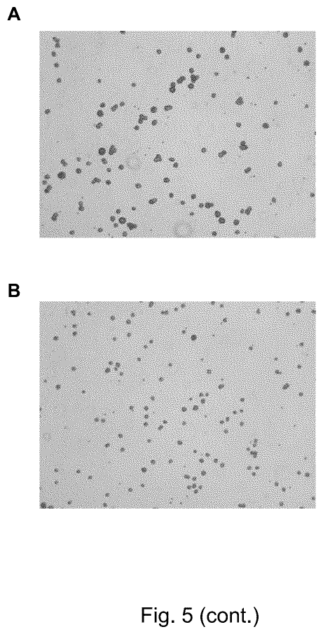

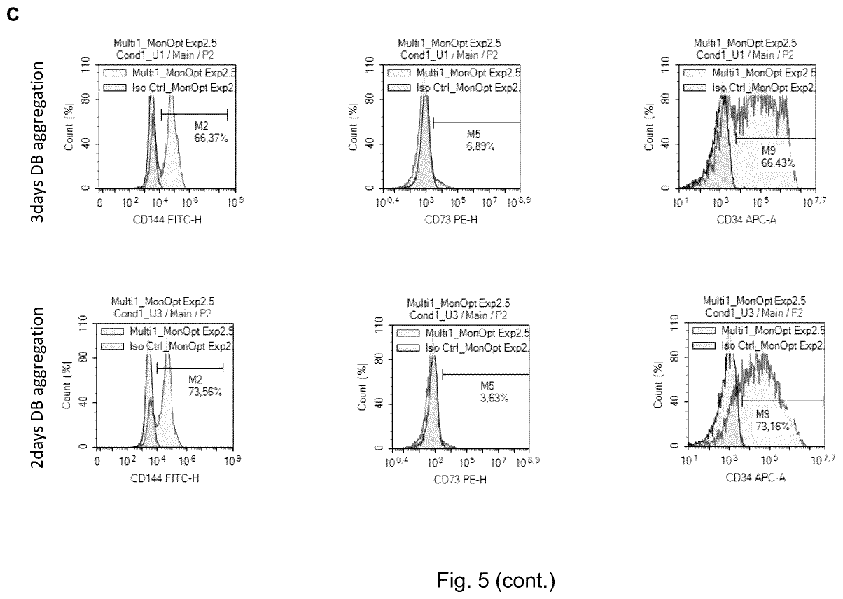

Epithelial cell derived iPSC were allowed to form aggregates for either 2 or 3 days prior to generation of HECs. Cells were inoculated in Das Box (DB; 40 M vc/unit). Average first aggregate size in diameter for cells allowed to form aggregates for 3 days (D-3-D0) was about 53.5 micrometer (FIG. 5A). Average first aggregate size in diameter for cells allowed to form aggregates for 2 days (D-2-D0) was about 40.5 micrometer (FIG. 5B).

After formation of a plurality of first cell aggegates comprising the PSCs, HE induction (D0-D6) was performed in 6wp suspension (5000 EB/well in 2 mL medium; refresh 100%). Medium for HE induction was according to Feng et al. (WO 2020/086889 A1, referred to herein), with one adaption, namely that 0.33 microM CHIR99021 was used.

### Results:

FIG. 5C shows measurements of CD144/CD73/CD34 markers in HEC obtained from 3 days DB (40 M vc/unit) aggregate formation (D-3-D0) and 2 days DB (D015%; 40 M vc/unit) aggregate formation (D-2 to D0). FIG. 5B shows that a smaller average first aggregate size (i.e. 40.5 micrometer (D-2-D0)) improves preferential HEC marker expression CD34 and/or CD144, and at the same time reduce the marker CD73.

### Example 3—BMP4 Dosing in HEC Differentiation

### Method:

Epithelial cell derived iPSC were allowed to form aggregates for 2 days in accordance to Example 2. First cell aggregates were induced for HEC formation for 6 days (D0-D6) in 6wp suspension (5000 EB/well in 2 mL medium; refresh 100%). iPSCs were exposed to different HEC culture media compositions.

On D0 the media composition was IF9S medium comprising BMP4 (in an concentration according to Table 1), VEGF in a concentration of 25 ng/ml, bFGF in a concentration of 25 ng/ml.

On D2 media were 100% refreshed and media provided to the cells on D2 was IF9S medium comprising BMP4 in a concentration according to Table 1, VEGF 25 ng/ml, bFGF 25 ng/ml and 3 μM CHIR99021. On D3 culture media were 100% refreshed and media provided to cells were identical to those provided on D2 and further comprising 3 μM SB431542. On D4 media were 100% refreshed and media provided to cells were identical to those provided on D3 but did not comprise 3 μM CHIR99021. On D5 media were 100% refreshed and media provided to cells were identical to those provided on D4, but did not comprise 3 μM SB431542.

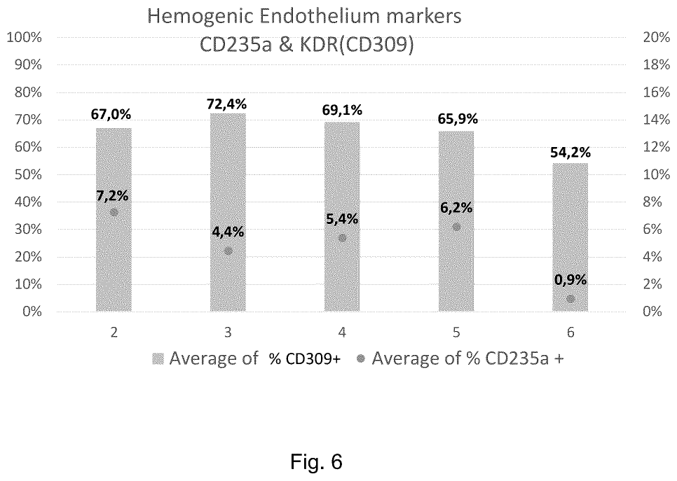

TABLE 1
BMP4 conditions (different BMP4 concentration over time)
Condition #'s correspond to those in FIGS. 6-9.

| Cond. # | BMP4 conc. D 0→D 2 | BMP4 conc. D 2→D 6 |
|---|---|---|
| 2 | 25 ng/ml | 25 ng/ml |
| 3 | 100 ng/ml | 100 ng/mL |
| 4 | 10 ng/ml | 10 ng/ml |
| 5 | 100 ng/ml | 25 ng/ml |
| 6 | 25 ng/mL | 0 ng/ml |

### Results:

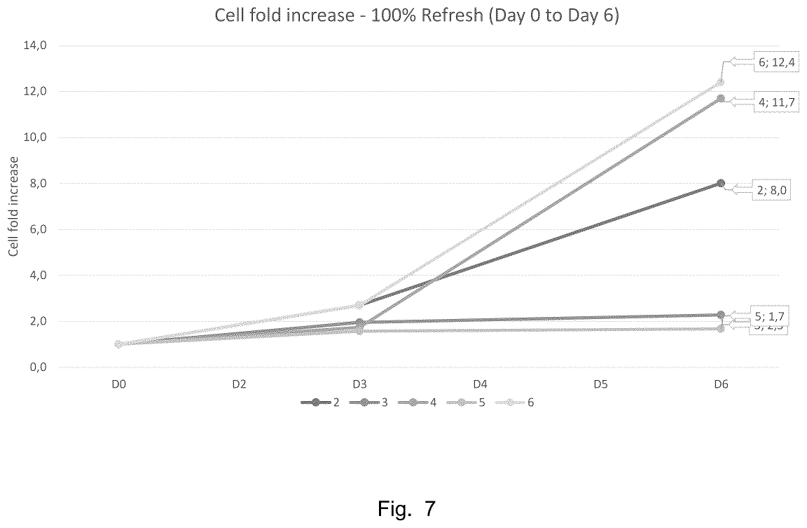

Results are shown in FIGS. 6-9. As shown in these figures, the condition containing 25 ng/mL BMP4 between D0-D2 and 0 ng/ml between D2-D6 resulted in a lower CD235 expression in comparison to the other conditions tested (FIG. 6) and resulted in a good yield (i.e. high cell fold increase) within 6 days (FIG. 7).

It was found that when BMP4 was provided (in any concentration) to the cells from Day2 this correlates with a higher CD235 expression (primitive marker) compared to no BMP4 (FIG. 6).

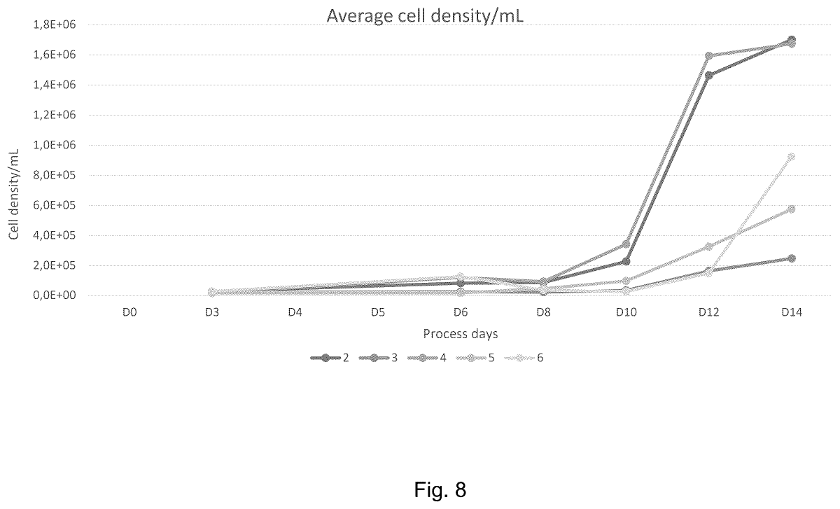

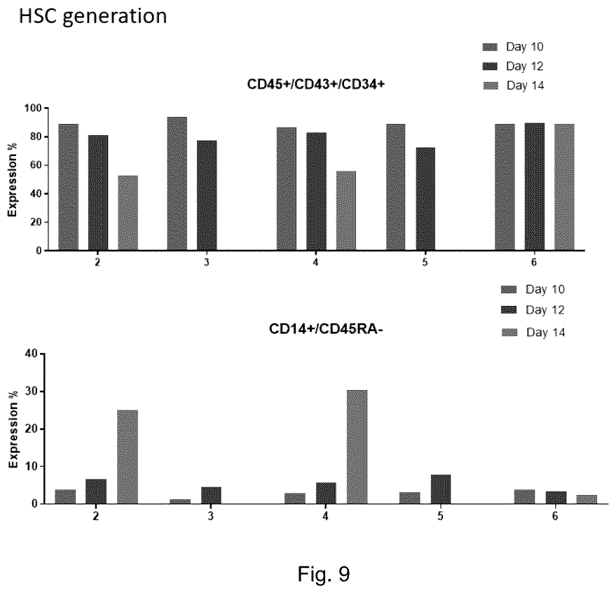

Moreover, condition 3, comprising a higher BMP4 concentration (100 ng/ml) on both D0-D2 and D2-D6 resulted in poor yield on D6 and a reduction in yield of HSCs (as evidenced by total HSC density/mL (FIG. 8) and by marker expression CD45/CD43/CD34 (FIG. 9)).

Further, it was found that in case BMP4 was provided to the cells from Day 2, even at low concentration (10 ng/ml) there was expression of myeloid marker (CD14) in HSC at Day 14 (FIG. 9), meaning that cells proliferated to myeloid cell line type of cells (i.e. expressing CD14+ and being negative for CD45RA).

BMP4 in the later stages of HE induction (e.g. from D2-D6 after HE induction) of iPSCs affects the lineage-specificity of the obtained population of HSCs. Hence, controlling concentrations of BMP4 allows pushing iPSCs and/or HECs to differentiate directly towards a specific lineage (e.g. lymphoid or myeloid).

### Example 4—HEC to HSC Differentiation

Epithelial cell derived iPSC were allowed to form aggregates for 2 days in accordance to Example 2 and allowed to form HECs according to Example 3, wherein cells were exposed to BMP4 levels in accordance to condition 6 of Table 1.

### Example 4.1. bFGF

Various media for HSC generation (D6-D14) in 6wp suspension (3 mL medium; refresh 66,67%) in accordance to Table 2.

TABLE 2
Different media compositions for HSPC generation

| HEC-HSC | | BSA | ITS-X | Bmercapto | AA2P | Glutamax | Pen/strep | hSCF | TPO | FLT3I | IL-3 | IL-6 | bFGF |
|---|---|---|---|---|---|---|---|---|---|---|---|---|---|
| Medium | IMDM | (%) | (%) | (μM) | (μg/mL) | (mM) | (U/mL) | (ng/mL) | (ng/mL) | (ng/mL) | (ng/mL) | (ng/mL) | (ng/mL) |
| Medium 1 | Y | 0.2 | 1 | 50 | 50 | 2 | 50 | 50 | 50 | 50 | 10 | 10 | — |
| Medium 2 | Y | 0.2 | 1 | 50 | 50 | 2 | 50 | 50 | — | 50 | — | — | — |
| Medium 3 | Y | 0.2 | 1 | 50 | 50 | 2 | 50 | 50 | 50 | 50 | 1 | 10 | 10 |
| Medium 4 | Y | 0.2 | 1 | 50 | 50 | 2 | 50 | 50 | — | 50 | — | — | 10 |

On day 14 HSPCs were analyzed for purity and yield.

### Results

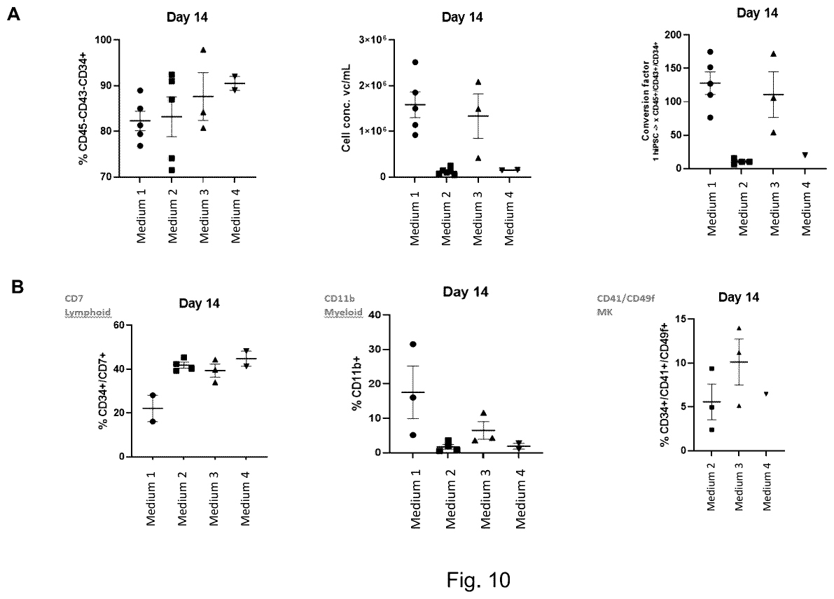

For all four media tested the average purity of cells expressing the expression markers CD45+/CD43+/CD34+was higher than 80% (FIG. 10A).

Medium 2 (i.e. Matsubara-like medium (Matsubara H. et al. “Induction of human pluripotent stem cell-derived natural killer cells for immunotherapy under chemically defined conditions” Biochem Biophys Res Commun. 2019 Jul. 12;515 (1): 1-8)) resulted as expected in a poor yield HSC medium, in comparison to Medium 1 or Medium 3 (FIG. 10A). However, it was found that Medium 4 (FIG. 10A) improves yield (2-3,6 fold higher than comparative Medium 2 HSC medium in respective experiment). Medium 2, 3 and 4 provided for high CD7 expression in the CD34+ population of resulting HSC thus providing for a lymphoid bias. Medium 1 allowed for higher presence of the myeloid marker CD11b. (FIG. 10B)

### Example 4.2—bFGF & DLL4

Various media for HSC generation (D6-D14) in 6wp suspension (3 mL medium; refresh 66,67%) and with or without coating DLL4 FC. Conditions tested are shown in Table 3 & 4:

TABLE 3
HSPC culture media

| HSC | | BSA | ITS-X | Bmercapto | AA2P | Glutamax | Pen/strep | hSCF | TPO | FLT3I | IL-3 | IL-6 | bFGF |
|---|---|---|---|---|---|---|---|---|---|---|---|---|---|
| Medium | IMDM | (%) | (%) | (μM) | (μg/mL) | (mM) | (U/mL) | (ng/mL) | (ng/mL) | (ng/mL) | (ng/mL) | (ng/mL) | (ng/mL) |
| Medium A | Y | 0.2 | 1 | 50 | 50 | 2 | 50 | 50 | — | 50 | — | — | — |
| Medium B | Y | 0.2 | 1 | 50 | 50 | 2 | 50 | 50 | 50 | 50 | 1 | 10 | 10 |
| Medium C | Y | 0.2 | 1 | 50 | 50 | 2 | 50 | 50 | — | 50 | — | — | 10 |

TABLE 4
HSPC generation conditions

| | | | | HSPC | HSPC |
|---|---|---|---|---|---|
| | Aggregate | HEC | | generation - | generation - |
| Condition | formation | formation | HSC medium | Coating | shaker |
| 1 | 2-day DB | 2-day | Medium B | — | 60 rpm |
| | | BMP4 | | | |
| 5 | 2-day DB | 2-day | Medium A | 5 μg/ml | 0 rpm |
| | | BMP4 | | DLL4 | |
| 10 | 2-day DB | 2-day | Medium C | | 60 rpm |
| | | BMP4 | | | |
| 11 | 2-day DB | 2-day | Medium A | — | 60 rpm |
| | | BMP4 | | | |

### Results:

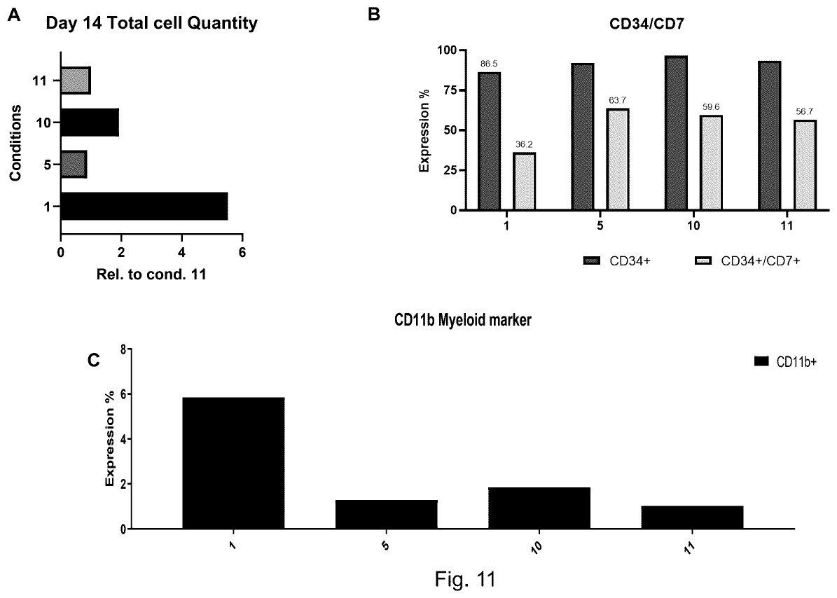

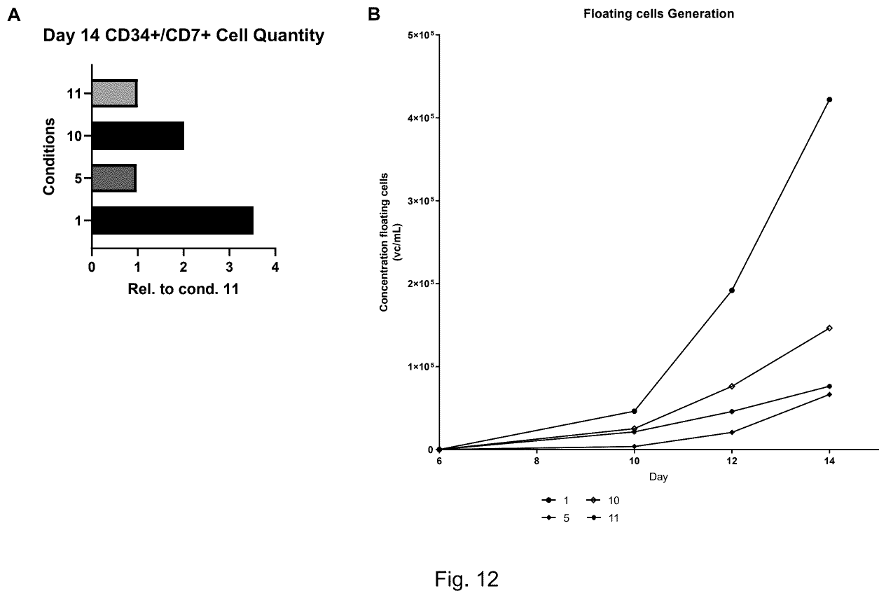

Results are shown in FIGS. 11A-C and 12A-B.

### Example 4.3—IL-3

PSCs were differentiated using the 2-day BMP4 protocol as described for condition 6 in example 3. HSC were generated under various conditions (see Table 5) using a medium comprising IMDM, 0.2% BSA, 1% ITS-X, 50 UM beta-mercaptoethanol, 50 μg/ml AA2P, 2 mM Glutamax, 50 U/ml pen/strep, 50 ng/ml hSCF, 50 ng/ml TPO, 50 ng/ml FLT31, 10 ng/ml IL-6 (HSC med Ctrl), supplemented with any supplement mentioned under “HSC generation” in Table 5 (e.g. condition 2, 3, 12, 13) or IMDM, 0.2% BSA, 1% ITS-X, 50 μM beta-mercaptoethanol, 50 μg/ml AA2P, 2 mM Glutamax, 50 U/ml pen/strep, 50 ng/ml hSCF, 50 ng/ml FLT3I (Basal medium 2+SCF +FLT31-condition 10).

TABLE 5
HSC generation conditions

| Conditions # | HE induction | HSC generation |
|---|---|---|
| 2 | 2-day BMP4 | HSC med Ctrl |
| | | complete |
| 3 | 2-day BMP4 | HSC med Ctrl with 1 |
| | | ng/mL IL-3 |
| 10 | 2-day BMP4 | Basal Medium 2 + |
| | | SCF + FLT3I |
| 12 | 2-day BMP4 | HSC med Ctrl |
| | | complete + |
| | | coating retronectin − |
| | | DLL4-Fc |
| 13 | 2-day BMP4 | HSC med with 1 |
| | | ng/ml IL-3 + |
| | | bFGF 10 ng/ml |

### Results

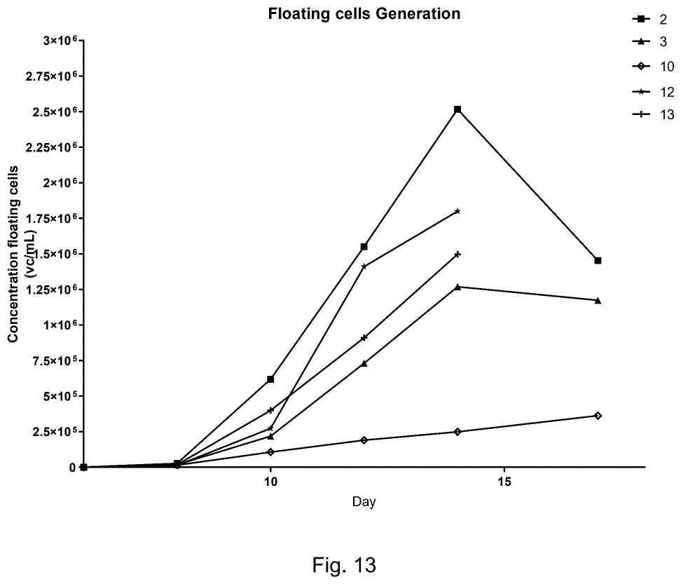

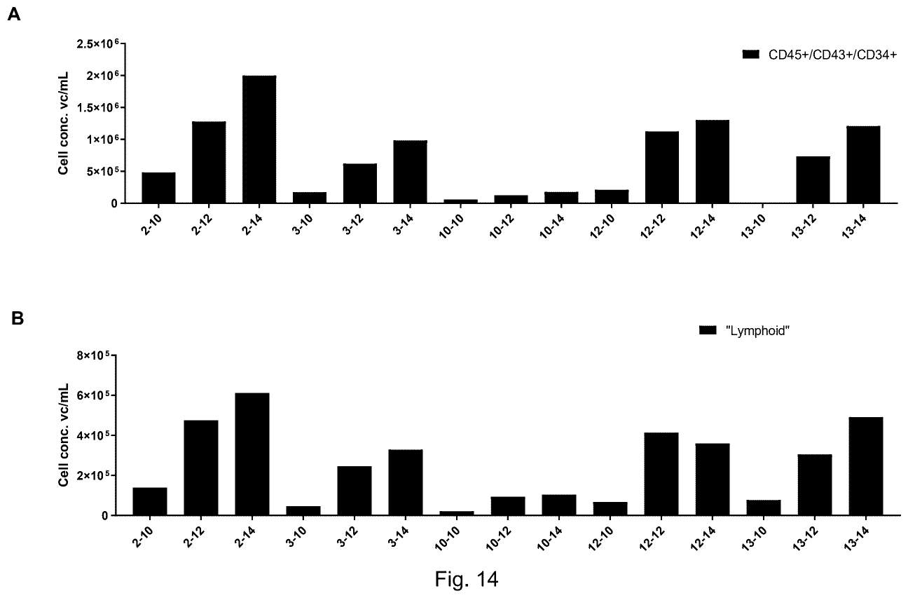

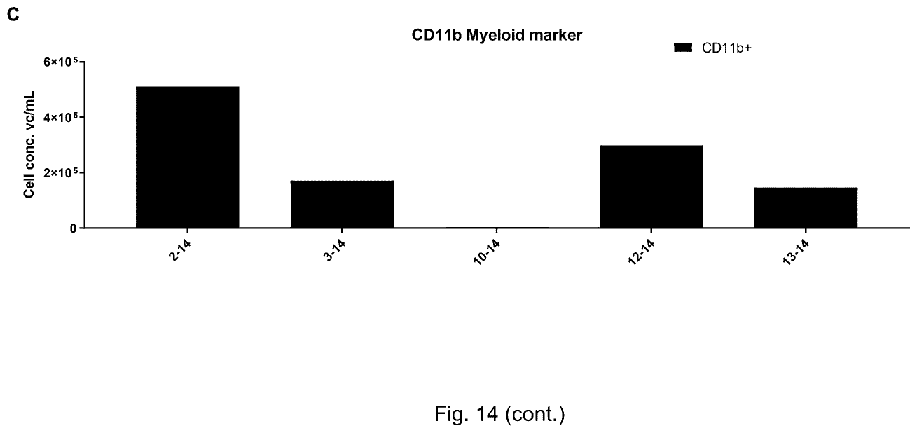

Results are shown in FIGS. 13 and 14. In FIG. 13, when comparing condition 2 to condition 12, there is an effect of DLL4 coating in the amount of floating HSPCs generated (2,5 mln/mL vs 1,8 mln vc/mL). Moreover, when comparing condition 3 vs condition 13 the effect of bFGF addition is that there is an increase in amount of floating HSPCs by addition of bFGF (1,3 mln/mL vs 1,5 mln vc/mL).

In FIG. 14A the DLL4 coating in condition 12 resulted in lower cell concentration of cells expressing CD45/CD43/CD34 on Days 10, 12 and 14 compared to condition 2. Moreover, the effect of bFGF addition (condition 13) vs. condition 3 is also an increase in cells expressing CD45/CD43/CD34 (FIG. 14A). This is in line with lymphoid expression markers for these conditions in FIG. 14B, wherein addition of bFGF results in an increase in CD7+ cells (condition 13). Hence it was found that lowering concentration of IL-3 (1 ng/ml), i.e. conditions 3 and 13, maximizes lymphoid progenitor production (FIG. 14B CD7+) and/or minimizes myeloid progenitors production (FIG. 14C CD11b+).

### Example 4.4—Post-Thawing Supplementation

HSC generation (D6-D14) in 6wp suspension (refresh 66,67%) and by differentiating from HECs from D6-D14 using a medium comprising IMDM, 0.2% BSA, 1% ITS-X, 50 μM beta-mercaptoethanol, 50 μg/ml AA2P, 2 mM Glutamax, 50 U/ml pen/strep, 50 ng/ml hSCF, 50 ng/ml TPO, 50 ng/ml FLT31, 10 ng/ml IL-6 and 10 ng/ml IL-3.

HSCs were frozen and thawed, thereby using post-thawing media compositions comprising IMDM, 0.2% BSA, 1% ITS-X, 50 UM beta-mercaptoethanol, 50 μg/ml AA2P, 2 mM Glutamax, 50 U/ml pen/strep, 50 ng/ml hSCF, 50 ng/ml TPO, 50 ng/ml FLT31, 10 ng/ml IL-6 and optionally supplemented with 10 ng/ml IL-3, 1 ng/ml IL-3 and/or 1 μM UM729. Supplementing of the post-thawing media compositions with IL-3 and/or UM729 resulted in certain conditions, shown in Table 6.

TABLE 6
Post-thawing conditions

| Condition # | Supplement |
|---|---|
| 2 | 10 ng/ml IL-3 |
| 5 | — |
| 6 | 1 ng/ml IL-3 |
| 7 | 10 ng/ml IL-3 + |
| | UM729 1 μM |
| 8 | UM729 1 μM |
| 9 | 1 ng/ml IL-3 + |
| | UM729 1 μM |

Read-outs of current experiment are:
Facs at Day 0, 3 and at Day 7:3 panels (CD45/CD43/CD34, CD7, CD11b) for determining purity.
Proliferation from Day 0 to Day 7 for determining yield/quantity.

### Results

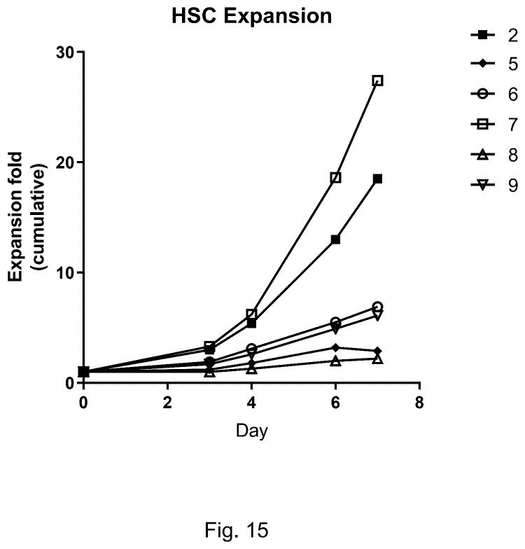

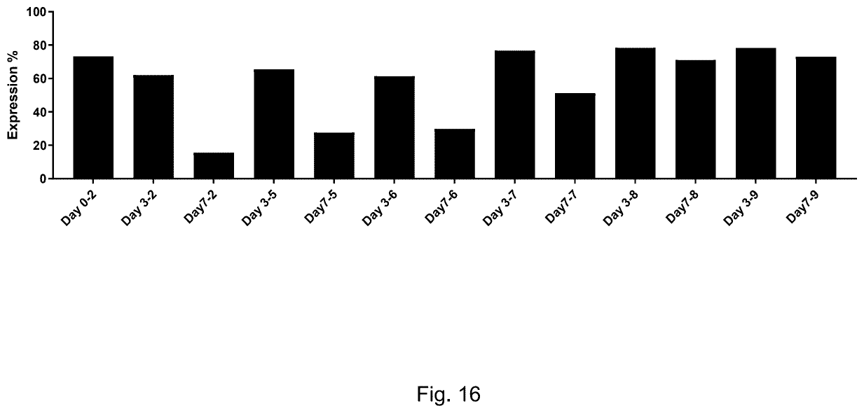

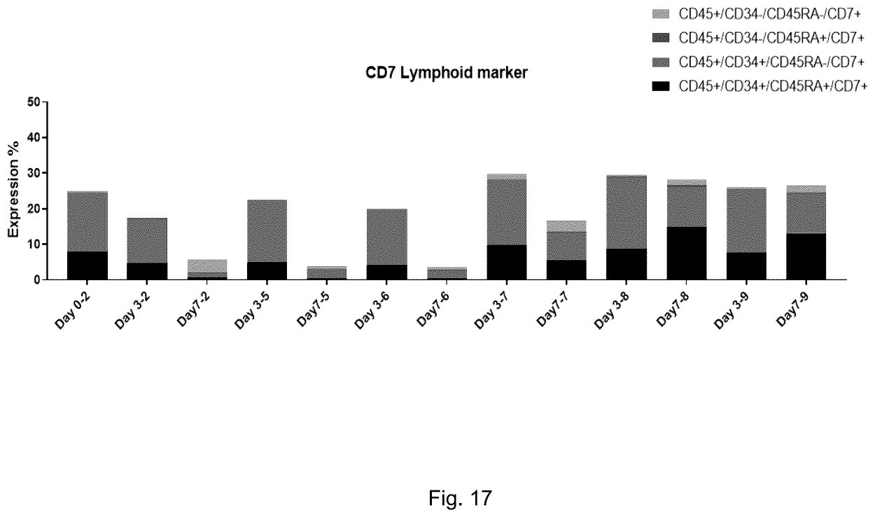

As shown in FIG. 15, the addition of UM729 1 μM (cond. 7) allows for higher expansion of HSC (CD45+/CD43+/CD34+) overtime post thawing. Moreover, in FIG. 16, it is shown that the overall post-thawing HSC profile is maintained (cfr day 3 data), and is a higher purity of HSCs (in % CD45+/CD43+/CD34+) observed in the case of addition of UM729 1 μM at Days 3 and 7 (e.g. when comparing cond. 2 vs 7, 5 vs 8, 6 vs 9) and overall by the addition of UM729 the HSC marker profile is better sustained over time (see day 3 to 7 data of cond. 7, 8 & 9). It is further shown that the highest purity is maintained when combining addition of UM729 1 μM (cond. 8 and 9) and low (e.g. 1 ng/ml) or no IL-3 at Days 3 and 7, for example compared to cond. 7 (10 ng/ml IL-3). However, as can be seen in FIG. 15, there is an impact of the reduction of IL-3 concentration on overall yield. In FIG. 17, CD7+ (lymphoid lineage marker) maintains stable expression on Days 3 and 7 for cond. 8 and 9 (no or low IL-3+UM729 1 μM).

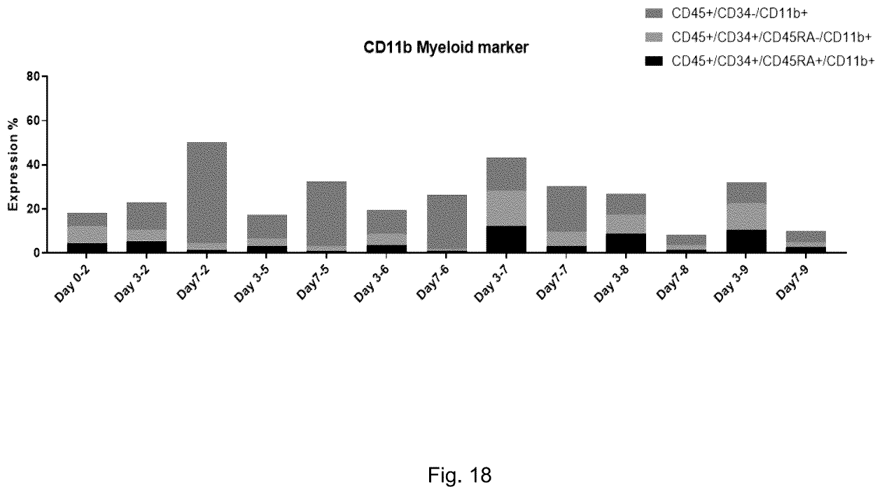

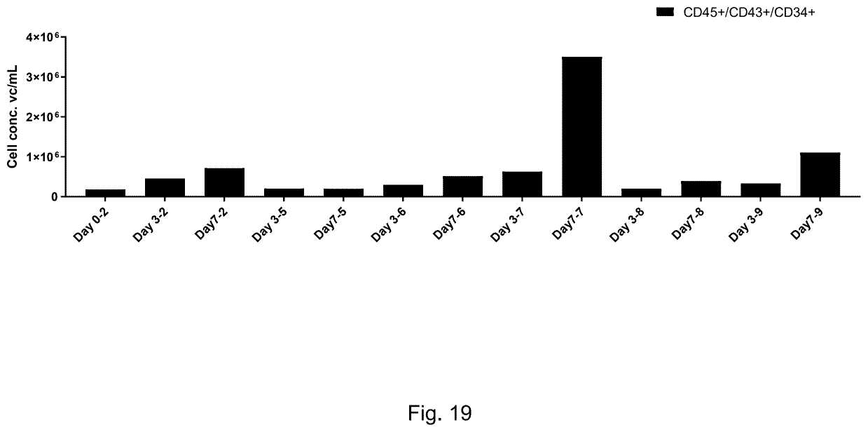

In FIG. 18 it is seen that on Day 7 there is a low CD11b (i.e. myeloid lineage marker) for both cond. 8 and 9 (no or low IL-3+UM729 1 μM). FIG. 19 shows that the addition of UM729 1 μM (cond. 7 (=Day 3-7 and Day 7-7)) allow for a higher production of CD45+/CD43+/CD34+overtime post thawing. Up to 3 million CD45+/CD43+/CD34+/mL in cond. 7 (medium M6) after starting with 250 k total cells/mL directly post-thawing.

Having now fully described this invention, it will be appreciated by those skilled in the art that the same can be performed within a wide range of equivalent parameters, concentrations, and conditions without departing from the spirit and scope of the invention and without undue experimentation.

Reference to known method steps, conventional methods steps, known methods or conventional methods is not in any way an admission that any aspect, description or embodiment of the present invention is disclosed, taught or suggested in the relevant art.

1. A method for in vitro production of a population of hemogenic endothelial cells (HECs), comprising:
(a) culturing a suspension of pluripotent stem cells (PSCs) thereby providing a plurality of first cell aggregates comprising said PSCs;
(b) culturing the plurality of first cell aggregates in culture medium to induce differentiation of the PSCs comprised in the plurality of first cell aggregates to generate a plurality of second cell aggregates comprising hemogenic endothelial cells (HECs), wherein
the plurality of first cell aggregates have an average size of about 49 or less micrometers in diameter; and/or,
wherein step (b) comprises a step (b1) of culturing wherein the culture medium does comprise a SMAD pathway agonist, preferably BMP4, and, a step (b2) of culturing wherein the culture medium is at least partly replaced by fresh medium which does not comprise a SMAD pathway agonist, and, wherein step (b1) is before step (b2).

2. The method of any of the previous claims, wherein the duration of step (b2) is more than 50% of step (b) and/or wherein the duration of step (b1) is less than 50% of step (b), preferably wherein step (b2) is for at least two, three or four days and/or wherein step (b1) is for at most three, two or one day.

3. The method of any of the previous claims, wherein the SMAD pathway agonist is BMP4 and wherein the concentration of BMP4 in the culture medium of step (b1) is between 10 and 40 ng/ml, preferably wherein the (residual) concentration of BMP4 in the culture medium of step (b2) is below 5 ng/ml.

4. The method of any of the previous claims, wherein between step (b1) and (b2) at least 70 vol. %, 80 vol. %, 90 vol. % or 95 vol. % of the medium is replaced by fresh medium.

5. The method of any of the previous claims, wherein during at least part of step (b2) cells are cultured in the presence of a TGFbeta/ALK inhibitor, such as SB431542, and/or, a GSK-3beta inhibitor, such as CHIR99021.

6. The method of any of the previous claims, wherein during step (b) the culture medium comprises VEGF and/or bFGF.

7. The method of any of the previous claims, wherein the plurality of first cell aggregates have an average size of about 20-250 micrometers in diameter, or have an average size of about 20-150 micrometers, preferably have an average size of about 20-55 micrometers or have an average size of about 25-50 micrometers or have an average size of about 30-40 micrometers.

8. The method of any of the previous claims, wherein the plurality of second cell aggregates have an average size of about 150 to 600 μm in diameter.

9. The method of any of the previous claims, further comprising step (c) where in step (c1) the plurality of second cell aggregates are cultured in a culture medium to induce differentiation of the HECs comprised in the plurality of second cell aggregates to generate a plurality of third cell aggregates producing hematopoietic stem cells (HSCs) and allowing the HSCs to release (through budding) from the plurality of third cell aggregates in the culture medium to obtain a population of HSCs thereby forming a single cell population comprising HSCs in suspension in the culture medium.

10. The method of any of the previous claims, wherein the plurality of first cell aggregates are provided in the first culture medium at a density of at least 100 aggregates/ml, preferably between 100-100000 aggregates/ml.

11. The method of any of the previous claims, wherein the method does not comprise a cell-selection or cell-separation step prior to optionally harvesting the single cell population comprising HSCs from the culture medium of step (c).

12. The method of any of the previous claims, wherein step (c) further comprises a step (c2) wherein the single cell population comprising the HSCs are separated from the remaining cell aggregates, and, step (c1) precedes step (c2).

13. The method of any of the previous claims, wherein the single cell population comprises at least 50% HSC cells, or at least 75% HSC cells, or at least 90% HSC, wherein the HSC cells express the marker(s) CD34, CD43 and/or CD45.

14. The method of any one of the previous claims, wherein the separation is done by counterflow centrifugal elutriation.

15. The method of any of the previous claims, wherein the culture medium to induce differentiation of the HECs comprises one or more growth factors selected from the group consisting of TPO (e.g. between 10 and 250 ng/ml, preferably about 50 ng/ml), hSCF (e.g. between 10 and 250 ng/ml, preferably about 50 ng/ml), FIT3-L (e.g. between 10 and 250 ng/ml, preferably about 50 ng/ml), IL-6 (e.g. between 2 and 50 ng/mL, preferably about 10 ng/ml), IL-3 (e.g. between 2 and 50 ng/ml), Wnt3a, bFGF, FICZ and TCDD.

16. The method of any of the previous claims, wherein the culture medium to induce differentiation of the HECs comprises bFGF in combination with one or more further growth factor initiating HSC differentiation and/or expansion, such as in combination with FLT3L and one or more further growth factor initiating HSC differentiation and/or expansion, and/or, such as in combination with SCF and one or more further growth factor initiating HSC differentiation and/or expansion.

17. The method of any of the previous claims, wherein
the HECs that are comprised in the plurality of second cell aggregates express the marker(s) CD34 and/or CD144; and/or
the plurality of second cell aggregates comprises:
i. at least 30% cells expressing CD34;
ii. at least 30% cells expressing CD144; and/or
iii. at most 30% cells expressing CD73.

18. The method of any of the previous claims, wherein
the HSCs released from the plurality of third cell aggregates, preferably wherein the HSCs are in the form of a suspension of single cells, express the marker(s) CD34, CD43 and/or CD45; and/or
the population of HSCs obtained in step (c) comprises:
i. at least 50% cells expressing CD34;
ii. at least 50% cells expressing CD43;
iii. at least 50% cells expressing CD45; and/or
iv. at most 30% cells expressing CD14.

19. The method of any of the previous claims, wherein the HSCs released from the plurality of third cell aggregates are cryopreserved to obtain cryopreserved HSCs.

20. The method of any of the previous claims, wherein the method comprises a step (d) that comprises further culturing the HSCs and/or the cryopreserved HSCs in culture medium to induce proliferation thereof.

21. The method of any of the previous claims, wherein the expansion ratio PSC: HSC is at least 50, at least 100, or at least 400.

22. The method of any of the previous claims, wherein the HECs and/or HSCs are further cultured to induce differentiation of the cells into lymphoid cells, such as lymphoid progenitor cells, T-cells (including alpha/beta T-cells and gamma/delta T cells), helper T (TH) cells, regulatory T (Treg) cells, natural killer (NK) cells.

23. The method of any of the previous claims, wherein the method is executed in a closed system or bioreactor, in particular wherein the method does not include a cell selection step or specific cell-type enrichment step.

24. The method of any of the previous claims, wherein the cell culture is a suspension culture without solid support or (exogenous) feeder cells.

25. Composition comprising a first population and a second population of cells, wherein the first population of cells is in the form of cell aggregates comprising HEC cells and wherein the second population of cells is in the form of a suspension of single cells comprising HSC cells, preferably wherein the second population comprises at least 50% HSC cells, or at least 75% HSC cells, or at least 90% HSC, wherein the HSC cells express the marker(s) CD34, CD43 and/or CD45, or wherein the composition comprises of the second population of cells.

26. A population of HSCs or cryopreserved HSCs as defined in any of the previous claims.

27. A population of HECs and/or HSCs and/or of differentiated cells selected from the group consisting of lymphoid cells, T-cells (including alpha/beta T-cells and gamma/delta T cells), helper T (TH) cells, regulatory T (Treg) cells and natural killer (NK) cells obtainable by the method of any one of claims 1-24.

28. A population of HSCs or cryopreserved HSCs characterized in that the population of cells comprises:
i. at least 70% cells expressing CD34;
ii. at least 70% cells expressing CD43; and/or
iii. at least 70% cells expressing CD45,
and wherein the percentage of cells expressing CD34, CD43 and CD45 remains stable when the cells are proliferated for one or two days according to step (d).

29. A culture system or a bioreactor, preferably a closed culture system, with culture medium comprising the cells or population of cells of claims 26-28, or comprising cells or cell aggregates as defined in the method of any of claims 1-24.
